(19) United States
(12) Patent Application Publication
(10) Pub. No.: US 2008/0281425 A1
(43) Pub. Date: Nov. 13, 2008

(54) Orthopaedic Implants and Prostheses

(76) Inventors: John Thalgott, Las Vegas, NV (US); David T. Stinson, Woodinville, WA (US)

Correspondence Address: Beusse Wolter Sanks Mora & Maire, 390 N. ORANGE AVENUE, SUITE 2500, ORLANDO, FL, 32801, US

(21) Appl. No.: 12/034,062

(22) Filed: Feb. 20, 2008

Related U.S. Application Data
(60) Provisional application No. 60/890,923, filed on Feb. 21, 2007.

(51) Int. Cl. A61F 2/44

(52) U.S. Cl. 623/17.16

(57) ABSTRACT

Disclosed herein are modular spinal implants having components which are interlocked together to form a single implant. Specifically exemplified herein are implants that are sectioned along a longitudinal plane. Implants are disclosed which include channels for inter-fragmentary association with an elongate bone screw and which allow for angular variability of the screw relative to the channel. Also disclosed is an anti-backout mechanism that helps prevent fixators from backing out upon securement of the implant in the spine. Kits comprising different sizes and inclination angles of components are disclosed, which can assist the surgeon in preoperatively assembling an implant to best fit in the surgical site of the patient.

### CROSS REFERENCE TO RELATED APPLICATIONS

The present application claims priority to U.S. provisional patent application Ser. No. 60/890,923 filed Feb. 21, 2007, whose teachings are incorporated by reference.

### FIELD OF THE INVENTION

The present invention relates to orthopaedic implants and/or prostheses and instrumentation for their implantation. The invention is applicable to bone structures, particularly the cervical, thoracic and lumbar spine.

### GENERAL BACKGROUND

Spinal fusion for the management of lumbar degenerative disc disease has been available for several decades. The results of this procedure remain under constant scrutiny and progressive development. Anterior lumbar fusion was initially introduced in the early 1920s. Fibula and iliac struts, femoral rings and dowel, as well as synthetic metallic devices have been applied as fixation implements to aid in lumbar interbody fusion. Approaches to the spine have experienced similar evolutionary changes. Prior to the 1950s most anterior lumbar approaches were extensive transperitoneal exposures (i.e. through the membrane lining the walls of the abdominal and pelvic cavities). In 1957, Southwick and Robinson introduced the retroperitoneal approach (i.e., behind the peritoneum). Transperitoneal exposures (i.e., through the peritoneum) require incision of both the anterior and posterior peritoneum. In contrast, retroperitoneal exposures maintain the integrity of the peritoneum and approach the spinal column laterally behind the bowel and peritoneal contents. This has the advantage of less post-operative bowel problems. Additional changes in technique have seen the advent of minimally invasive approaches, including endoscopic and laparoscopic methods. Minimally invasive approaches are generally directed at one or two-level disease processes. Anterior lumbar interbody fusion (ALIF) may be useful in the treatment of unyielding low-back pain. The cause of this pain is often difficult to diagnose. Broad categories of pathology that may be associated with persistent low-back pain include degenerative disc disease, spondylolysis, spondylolisthesis or iatrogenic segmental instability.

Bones and related structural body parts, for example spine and/or vertebrae and/or intervertebral discs, may become crushed or damaged as a result of trauma/injury, or damaged by disease (e.g. by tumour, auto-immune disease), or damaged as a result of degeneration through an aging process. In many such cases the structure can be repaired by replacing the damaged parts (e.g vertebra and/or discs) with a prosthesis or implant. A method of repair is to remove the damaged part(s) (e.g. vertebra and/or partial vertebra and/or disc and/or partial disc) and replace it with the implant or prosthesis such that the implant or prosthesis is free standing or fastened in position between adjacent undamaged parts (e.g adjacent vertebrae).

Associated with this method of repair, is fusion of the bone structure where the implant or prosthesis is placed. Typically an implant or prosthesis may consist of a central space surrounded by a continuous wall that is open at each end (e.g. superior and inferior). This form of implant or prosthesis is thought to allow bone to develop within the central space, developing from each extremity of the implant or prosthesis towards the centre. Typically an implant or prosthesis shall be secured directly to a bone structure by mechanical or biological means. Conventional implants pertain to solid materials typically taking the form of a dowel or general wedge shape that may be positioned in a bored hole or rammed into an intervertebral space. While there has been an evolution of the shape of implants and some attempts to provide modular implants, the inventors have recognized that such changes have been relatively minor and have not fully contemplated cooperation between optimizing the surgical result and improving efficiency and safety of the operative procedure.

### General Description

The subject invention is based on the inventors' recognition that conventional spinal implants and techniques possess several shortcomings not known by those in the art. The inventors have developed not only spinal implants that are superior in their design, but also have developed a comprehensive system for spinal surgery, including implants that are especially adapted for an anterior approach, lateral approach, and the rarely implemented anterolateral surgical approach. FIG. 33 illustrates the basic direction of access to the intervertebral space. The anterior approach comprises an approach directly from the anterior vector of the vertebral body with 20 degree variability, the anterolateral approach is 45 degrees from the anterior vector with 25 degree variability and the lateral approach is 90 degrees from the anterior vector with 20 degree variability. Implant embodiments of the present invention facilitate easier, quicker and more precise surgical techniques that enable the restoration and re-establishment of spinal anatomy, lordosis and/or disc height. Implant embodiments of the present invention also are safer to use and increase the chances of a positive surgical outcome.

A problem arises particularly with spinal implants and prostheses, because the size of the space into which the implant or prosthesis is to be inserted varies from patient to patient and also depends on its position in the bone structure e.g. the spinal column. In the case of conventional and commonly used single-piece implant such as dowel shaped implant (discussed in U.S. Pat. No. 6,033,438) or wedged shaped implant such as that described in U.S. Pat. No. 5,425,772, one solution to this problem is to have multiple shapes and sizes of implant or prosthesis. However, this results in intra-operative complexity and a large, hence expensive, range of inventory. Another solution to this problem is to have an implant with adjustable height. This adjustable height may be achieved through, for example, mechanical, hydraulic or pneumatic means. There are various designs with adjustable height on the market or described in literature, such as the use of dampers e.g. springs (Intervert Locking Device, described in U.S. Pat. No. 5,360,430), or a compressible core (Trieu—Compressible Corpectomy Device, described in U.S. Patent Publication 2005096744) or the use of liquids (Barber Vertebral Body Prosthesis, described in U.S. Pat. No. 5,236,460), or the use of stackable building blocks (DePuy Stackable Cage described in U.S. Pat. No. 6,159,211), or the use of adjustment by a screw principle (Berry VBR US2004186569).

Embodiments of the invention have an advantage over existing implants or prostheses in that their clinical use is simplified over current practice, resulting in shorter operative times, less risk to the patient and less cost. Embodiments described herein enable the intraoperative (intradiscal) assembly of components of a modular implant in the intervertebral space. In particular embodiments, implant configurations are provided that facilitate intraoperative assembly for implementation for the anterior, anterolateral and lateral surgical approaches. In certain embodiments, the components are configured such that they are sectioned and associate along a longitudinal plane, as illustrated in FIGS. 33 to 36. FIGS. 33 to 35 show that the modularity of the implants may be defined along a coronal plane CLP, which is particularly advantageous for anterior or lateral surgical approaches; and a transverse plane TP, which is particularly advantageous for an anterolateral surgical approach. Unless specifically stated otherwise, use of the term “longitudinal plane” to describe modularity of embodiments of the invention refers to sectioning along a coronal or transverse plane or a plane having at least a coronal or transverse aspect thereto.

In a specific embodiment, a first component is surgically placed into the intervertebral space at a predominantly posterior position then a second component is placed in a predominantly anterior position of the intervertebral space. Typically, this will be performed following measurement with trial spacers. The ability to first position a component posteriorly and then anteriorly enables the surgeon to intraoperatively optimize the size and slope of the implant for a patient's given anatomical size. This avoids the need for an unnecessarily large amount of different single piece sizes. The embodiment also accomodates a broad range of different space sizes and unique patient anatomy with a manageable set of component sizes. Furthermore, the placement of a predominantly posterior component followed by a predominantly anterior component facilitates the adjustment of lordosis as a function of the first component having a first size and dimension that serves as an initial support and forms the desired angle and space for placement of the second component having different size and dimension. Embodiments of the present invention are sectioned and configured to increase ease of insertion into the intervertebral space for each of the surgical approaches (anterior, anterolateral and lateral) while facilitating the interdiscal assembly of the implant. While the implant embodiments enable intraoperative assembly, those skilled in the art will appreciate that presurgical assembly of the components may be conducted dependent on the surgeon's preference.

Another problem recognized by the inventors involves the way that conventional implants interact with bone surface of the vertebral body. Many conventional implants with single piece or modular arrangement fail to take into account the natural anatomy of the interior surface of the vertebral body. The inventors are of the belief that maximizing the surface between the implant and vertebral body will improve the surgical result. Accordingly, in another embodiment, both the first and the second components comprise geometric dimensions that serve to restore anatomy, proper lordosis and/or disc height. In a particular embodiment, the individual components are assembled together to form a unitary implant that has a tapered convex shape in a sagital plane and may also be an elliptical shape in a coronal plane. This is an advantageous feature of the embodiments because, unlike conventional modular implants that lack a coordination of the components to form a geometric configuration mirroring the intervertebral space, the components of this embodiment increase implant/bone load bearing surface area, restore natural anatomy of the disc and establish a desired space height and a desired lordosis.

The inventors have recognized another problem associated with conventional spinal implants relating to the mode of securement of the implant to the vertebral body. For example, U.S. Pat. No. 7,232,464 ('464 patent, assigned to Synthes) teaches a spinal implant that comprises a body portion and a plate portion that is inset to the body portion. The '464 patent teaches that the boreholes of the plate should be threaded such that a bone screw may be rigidly screwed into the implant The '464 patent is under the misapprehension that threading the screws into threads in the implant provides a preferred affixation. While not excluding the implementation of this type of affixation, the inventors take a contrary viewpoint concerning the mode of affixing the implant to the vertebral body and the association between bone, fixator (e.g., screw) and implant. Accordingly, in certain embodiments, as shown in FIG. 29 the inner walls of the channels of the implant are not affixed to the fixator, such as by threads or otherwise. The fixator freely passes through the channel and is screwed into the vertebral body. As the fixator is tightened, this pulls the implant toward the vertebral body. Thus, the implant is secured to the vertebral body in a fashion analogous to the concept of interfragmentary compression, which unifies the load path from the bone to the implant. It is the inventors' belief that this association between implant, fixator and bone is superior to that described in the '464 patent.

Another problem that the inventors have recognized with conventional implants is an absence of variability in the vector that the bone fixator (screw) may be directed for securement to the vertebral bodies relative to the angle of the implant. For example, the '464 patent described above discloses a number of boreholes through which the fixators are directed through (in this example secured to the boreholes via threads) such as described in FIG. 28. However, the vector of the fixator is static. That is, the bone screw cannot move relative to the vector of the borehole. The inventors have recognized that this is a shortcoming in conventional design. Adjacent to the spinal column is critical vasculature for the body which runs down along the anterior portion of the spine. Further, the spinal nerves extend out laterally from the spine. Thus, a challenge for spinal surgeons is avoiding such vital anatomical structures during surgery as well as securing the implant so as to minimize possible interference between the implant or fixators and the vital anatomical structures subsequent to surgery. Accordingly, another implant embodiment comprises channels that allow for angular variability in the vector of the fixator is desired. FIG. 31 illustrates the angular variability or dynamism of the fixator allowed by the channel. This angular variability now provides surgeons with a level of adjustability with respect to where the fixators are secured and the orientation and placement of the implant relative to the fixators. This in turn will enable the surgeon to place the fixators in such a way as to minimize disrupting or damaging vasculature and nerves, whether intraoperatively or post-operatively, as well as adapt to a patient's unique anatomy. Increased safety and improved surgical outcomes are achieved.

In a specific embodiment, the channels of the implant are configured such that a fixator comprises angular variability of 40 degrees (see angle Z shown in FIG. 31) or less, preferably 25 degrees or less, around a central axis of the respective channel. The central axis pertains to a vector running through the center of the channel.

In other embodiments of the invention, another problem associated generally with affixation in the spine is addressed: fixator back out. That is, after insertion into the vertebra, the fixator runs the risk of working loose and/or backing out of the vertebra. The consequence of backout or loosening of the implant or prosthesis includes loss of stability, potential risk to the patient and a separate costly operation. According to one embodiment, the subject invention pertains to an implant device that comprises an anti-backout means to prevent backout of fixators. The concept of “backing out’ is somewhat controversial, as some surgeons take the stance that it is a real phenomenon, while others think this is not a real risk. The inventors have realized that depending on the surgical site and the patient's anatomy, and surgeon preference, it may be beneficial to lock certain channels while keeping other channels unlocked. Thus, in certain implant embodiments, the anti-backout means pertains to a pivotable lock proximate to the channel opening. Each channel can be individually and independently closed following affixation of the fixator to bone. The fixators may be screws, pins, staples, darts, bollards or other suitable fixators. The ability of each channel to be individually locked provides options to surgeon depending on the placement of the implant and surgeon preference.

As already discussed above, a number of vital vasculatures and nerves are adjacent to and extend from the spine. The inventors have recognized that in circumstances where a portion of an implant protrudes from the intervertebral space this can cause a wearing down of vasculature over time. In extreme cases, this can result in a rupture of the vasculature and probable death. Accordingly, in certain embodiments, the implants are characterized as “no profile”, i.e., fully contained within the intervertebral space without protrusion. Prior art is either designed in such a way whereby the anterior portion protrudes out anteriorly from the intervertebral space such as the '464 patent, or otherwise is not configured to allow fixation into superior and inferior vertebral bodies. In certain advantageous embodiments of the invention, the implant is both no profile and allows bi-directional fixation.

Another challenge that spinal surgeons face stems from the relatively small, confined surgical window available for insertion of the implant or components thereof into the subject's body which makes insertion of the implant difficult. The inventors have addressed this problem by providing an instrument interface structure that is configured to interact with the implant during insertion thereof. The instrument may take the form of an inner shaft having a screw thread type engagement feature for engagement with a posterior portion of an implant, an intermediate hollow shaft for location relative to said posterior portion and around which is situated an outer sleeve having location features for location with an anterior portion of said implant. The arrangement being such as to allow the intermediate shaft to move axially and cause the anterior portion into contact and securement to the posterior portion before removal of the instrument.

In certain embodiments, bone ingrowth materials are implemented which may be disposed within various cavities defined in the embodiments, and/or used as coating the components. Bone ingrowth materials may comprise known bioactive materials including but not limited to BMP or other suitable growth factors, allograft bone with/without stem cell enrichment, calcium phosphate, and/or autograft bone. See U.S. Pat. Nos. 6,899,107 and 6,758,849 for general information on osteoinductive, osteoconductive and/or osteogenic materials and implants. Further, in alternate embodiments, bone ingrowth materials are made of solid materials such as, for example, cortical bone or coralline hydroxyapatite, which are pre-cut and pre-shaped are are conjoined with other implant components during assembly of the implant.

According to one embodiment, the invention pertains to a modular inter-body implant having first and second components. The implant is sectioned along a coronal or transverse longitudinal plane or a plane having at least a coronal or transverse aspect thereto. The first and second components have perimeter side surface, a top surface and a bottom surface. In one particular embodiment, the first component has at least two channels defined therethrough. At least one channel is defined according to a vector that begins at the implant perimeter side surface and transverses a plane of the implant top surface and at least one channel defined according to a vector that begins at the implant perimeter side surface and transverses a plane of the implant bottom surface. The channels are sized and configured such that an elongate bone fixator having, for example, a diameter of between 1 and 10 mm may separately pass through each of said at least two channels so as to allow for 40 degrees or less angular variability of said elongate bone fixator about a central axis of each of said first and second channels. The channels are configured so as to allow an interfragmentary association with said elongate bone fixator. That is, the channels allow a non-static association between the inner wall of the channel and the surface of the bone fixator. The implant also includes an instrument interface associated therewith. The instrument interface may be an interface receptacle defined in said unitary implant or an interface extension extending from said implant. The further includes two or more locking components movably affixed thereto and each proximate to at least one of said at least two channels such that said locking component can be shifted to block at least a portion of its proximate channel. In another particular embodiment, the first component has at least one channel defined therethrough defined according to a vector that begins at the implant perimeter side surface and traverses a plane of the implant top surface or implant bottom surface and the second component has at least one channel defined therethrough defined according to a vector that begins at the implant perimeter side surface and traverse a plane of the implant top surface or implant bottom surface.

The first and second components may be adjoined by numerous configurations including, but not limited to, spigot arrangement, tongue and groove arrangement, screw-type arrangement, dowel and hole arrangement and bayonet arrangement. These will be described in further detail below.

According to another embodiment, there is provided a kit of parts for use in assembling a spinal implant or prosthesis, comprising: a plurality of component members for insertion into an intervertebral space, the component members being of a range of sizes and/or shapes to suit different sizes/shapes of intervertebral space. The component members are configured to interconnect to form a suitable implant which takes into account the dimensions of the particular subject treated. One exemplary means for the engageable interconnection of component members comprises a mechanical joint such as a push or snap-fit connection.

In a specific embodiment, the invention pertains to a kit for facilitating spinal surgery comprising a plurality of first components having differing dimensions, each first component comprising a top surface and bottom surface and side perimeter surface; and a plurality of second components, each second component comprising a top surface, a bottom surface and a side perimeter surface. The first components are configured to adjoin to said second components.

In a specific kit embodiment, the first components have at least two channels defined therethrough, the at least two channels have at least one channel being defined according to a vector that begins at the implant perimeter side surface and transverses a plane of the implant top surface and at least one channel defined according to a vector that begins at the implant perimeter side surface and transverses a plane of the implant bottom surface.

In another specific kit embodiment, the first components comprise at least one channel defined therethrough defined according to a vector that begins at the implant perimeter side surface and traverses a plane of the implant top surface or implant bottom surface. The second components have at least one channel defined therethrough defined according to a vector that begins at the implant perimeter side surface and traverses a plane of the implant top surface or implant bottom surface.

According to a particular embodiment, the present invention pertains to a method for surgically implanting an implant into an intervertebral space between a superior and inferior vertebra. The method pertains to positioning into the intervertebral space a first component having a top surface and bottom surface and side perimeter surface. The first component is engaged to a second component having a top surface, a bottom surface and a side perimeter surface, wherein the first and second components when engaged form a unitary implant, and wherein the second component has at least one channel defined therethrough according to a vector that begins at said implant perimeter side surface and traverses a plane of the implant top surface or implant bottom surface. The at least one channel is sized and configured such that an elongate bone fixator having a diameter of, for example, between 1 and 10 mm may separately pass therethrough so as to allow for 40 degrees or less angular variability of the elongate bone fixator about a central axis of the at least one channel. An elongate bone fixator is secured through the at least one channel and into the superior vertebra or the inferior vertebra. At least a portion of a disc in said intervertebral space may be removed prior to position the first component. The method may further entail inserting a trial spacer into the intervertebral space to measure intradiscal anatomy prior to positioning said first component. In a particularly advantageous embodiment, the unitary implant is securable to a superior and inferior vertebral body while having no profile with respect to said intervertebral space. Furthermore, the implant may be sectioned along a coronal, longitudinal plane, transverse, longitudinal plane or sagital plane. In another particularly advantageous embodiment, the first and/or second component is delivered to the intervertebral space via a rail instrument associated with an instrument interface provided on said first and/or second component. The rail instrument may be curved to assist with access via an anterolateral surgical approach. The elongate bone fixator may have a drilling portion and a self-taping portion. The second component may have at least one locking component movably affixed thereto and proximate to said at least one channel such that said locking component can be shifted to block at least a portion of said at least one channel.

Optionally, the kit of parts provides a modularity of parts for use in assembling a spinal implant or prosthesis. Modularity is provided by, for example, increasing or decreasing dimensions, in the way the two or more components of the implant or prosthesis interact with each other or adjust in one or more planes. Having a range of implants or prosthesis that are modular in shape and form means that they can be combined with each other to provide the desired shape and size. In one embodiment, for example, the component members comprise asymmetrically configured segments that can be assembled together in a variety of numbers and orientations of segments to make up the implant. Alternatively, components may be constructed from pairs of oppositely-tapered half-component wherein the tapered portions overlap one another. The height and/or depth of the assembled structure may be adjusted by adjusting the extent of the overlap.

It is an advantage that the practitioner can select an appropriate size of components from the kit of parts to suit the particular size and shape of the space into which the implant or prosthesis is to be inserted. Not only do sizes vary from patient to patient, but also the size and shape of the space varies according to the location in the spine. Accordingly, depending on the size and/or shape of a intervertebral space, a practioner can choose a first component, such as an anterior component, having a certain size and/or dimension, and a second component, such a posterior component, having a certain size and/or dimension, to customize the overall size and shape of the unitary implant to produce an implant particularly suitable for the surgical space.

These and other features of the present invention will become more fully apparent from the following description and appended claims, or may be learned by the practice of the invention as set forth hereinafter.

### BRIEF DESCRIPTION OF THE DRAWINGS

FIG. 1A shows a perspective view of a disassembled modular spinal implant embodiment.

FIG. 1B shows a posterior view of an assembled modular spinal implant embodiment.

FIG. 1C shows a top planar view of an assembled modular spinal implant embodiment.

FIG. 1D shows an anterior side view of an assembled modular spinal implant embodiment.

FIG. 1E shows a side view of an assembled modular spinal implant.

FIG. 2 shows a fixator embodiment for affixing a spinal implant.

FIGS. 3A and 3B illustrate an instrument for the implantation of the device of FIG. 1 and two steps associated with implantation.

FIG. 3C shows a perspective view of a third step of surgically implanting an embodiment.

FIG. 4 shows an anterior view of an implant embodiment fixated between two vertebral bodies.

FIG. 5 shows a side view of an implant embodiment fixated between two vertebral bodies.

FIGS. 6a to 6d illustrate a height adjustable implant according to a further aspect of the present invention.

FIG. 7 is a top perspective view of another modular embodiment in a disassembled state particularly useful for an anterior approach.

FIG. 8 is a top perspective view of the embodiment shown in FIG. 7 in an assembled state.

FIG. 9 is a cross-sectional view of the embodiment shown in FIG. 8 taken along line A-A of FIG. 8.

FIG. 10 is an anterior-side perspective view 10a of an assembled modular with a bone screw embodiment inserted therethrough, and a rear-bottom perspective view of the embodiment 10b.

FIG. 11 is an anterior-side perspective view of a modular embodiment such as that shown in FIG. 10 with a plurality of bone screws inserted therethrough.

FIG. 12 is an anterior-bottom perspective view of the embodiment shown in FIG. 11.

FIG. 13 is a front-side perspective view of the embodiment shown in FIG. 11 secured to vertebrae.

FIG. 14 is a see-through view of the embodiment secured to vertebrae.

FIG. 15 is a top view 15a, front view 15b, front-side perspective view 15c, and side view 15d of a modular embodiment such as that shown in FIG. 11.

FIG. 16 is a top-side perspective view of a modular embodiment in a disassembled state particularly useful for a lateral surgical approach.

FIG. 17 is a side perspective view of the embodiment shown in FIG. 16 in an assembled state.

FIG. 18 is a bottom perspective view of the embodiment shown in FIG. 17.

FIG. 19 is a side view of the embodiment shown in FIG. 17 as it would be when secured between two vertebrae.

FIGS. 20a, 20b are side and side see-through view respectively of the embodiment shown in FIG. 17 secured between two vertebrae.

FIG. 21 is a top-side perspective view of a further embodiment of the present invention particularly suited to an anterio lateral approach.

FIG. 22 is a top end perspective view of the arrangement of FIG. 21.

FIG. 23 is a top view of the embodiment shown in FIG. 22 in an assembled state.

FIG. 24 is an anterior view of the embodiment shown in FIG. 22.

FIG. 25 shows a side perspective view of the embodiment shown in FIG. 22

FIG. 26 is a side view of the embodiment shown in FIG. 22.

FIG. 27 is a see-through front-top perspective view of the embodiment shown in FIG. 22 secured between two vertebrae.

FIG. 28 is a cross-sectional view of the implant shown in the above drawings and illustrating how different sized portions can be adjoined along the common plane.

FIGS. 29 to 31 illustrate the bone fixator and locking arrangement in more detail.

FIG. 32 is a cross-sectional view of a spine and illustrates the various surgical approaches associated with inter-vertebral repair.

FIGS. 33 to 36 show the various embodiments described above as they would be positioned in a spine.

FIG. 37 and FIG. 38 are cross-sectional views of superior and inferior vertebra and illustrate two methods of securing the implant described above to said vertebrae.

### DESCRIPTION OF ILLUSTRATIVE EMBODIMENTS

### EXAMPLE 1

### Anterior Approach

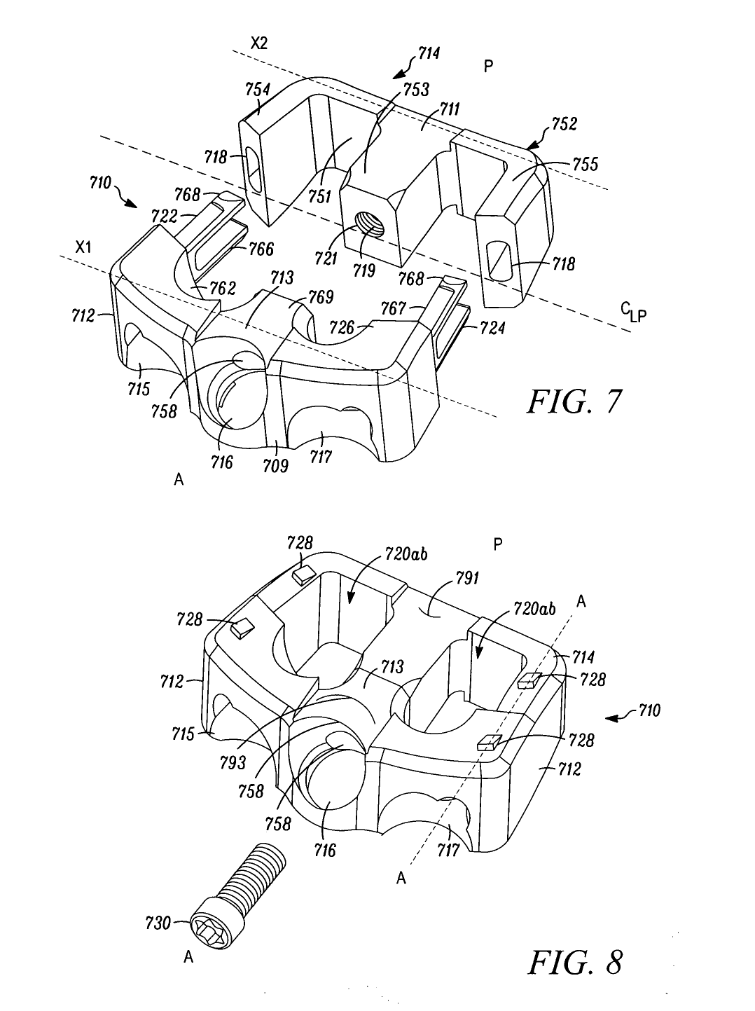

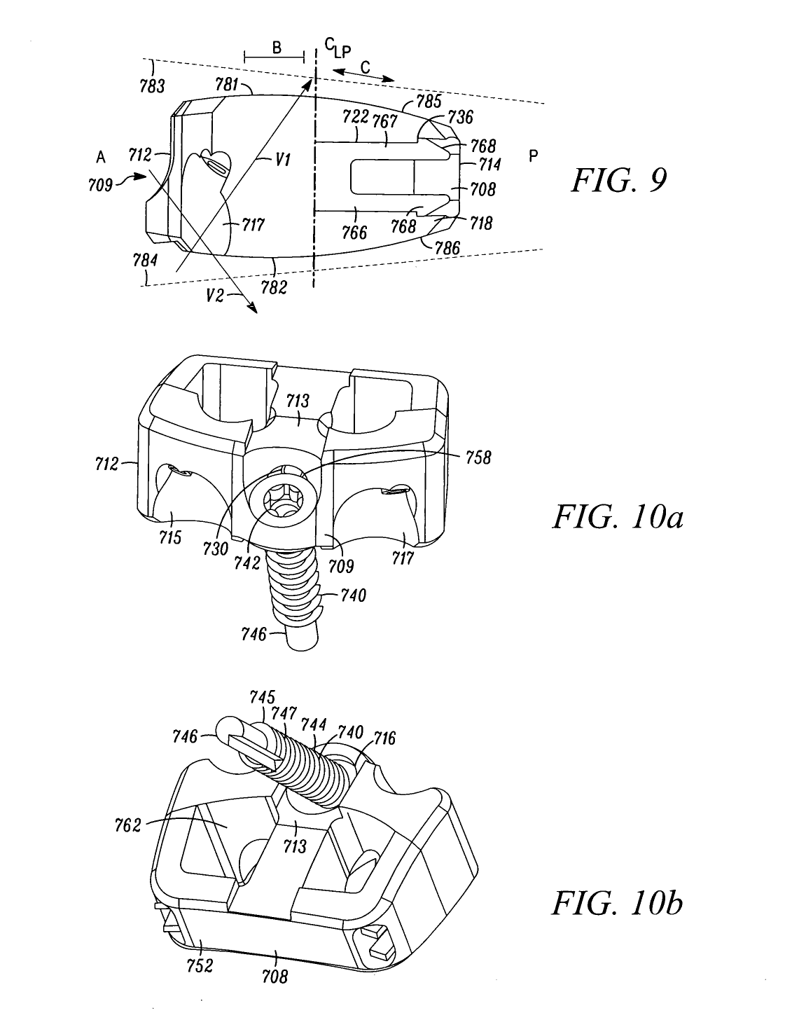

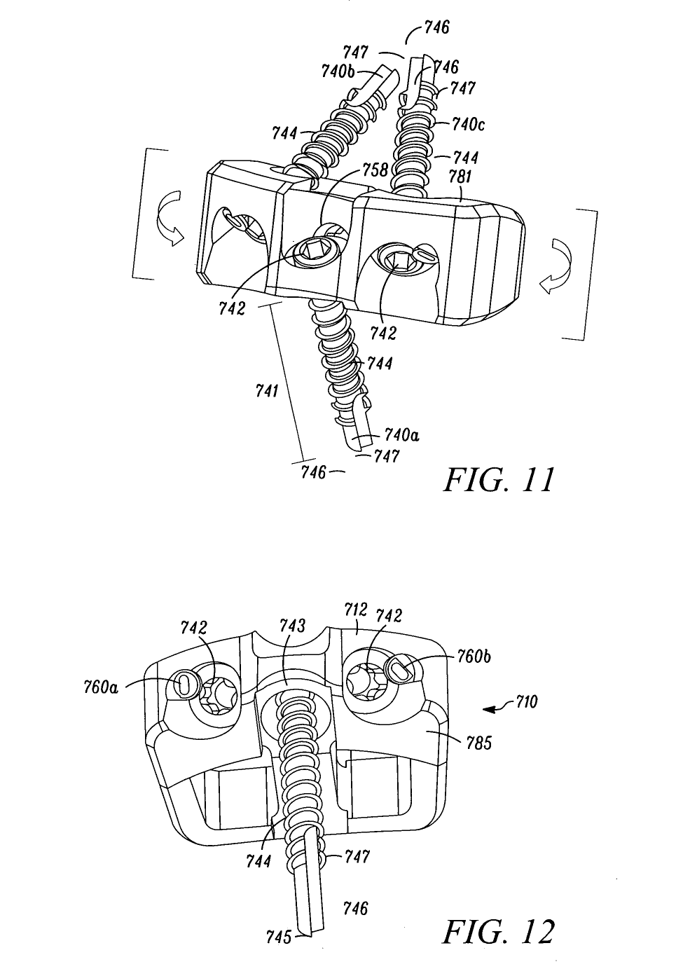

Reference to specific embodiments will begin with description of the embodiment as shown in FIGS. 7-15. According to this embodiment, the invention pertains to a modular implant 700 comprising an anterior component 712 and a posterior component 714. Modular implant 700 is sectioned along a coronal, longitudinal plane C_{LP} and is particularly useful for use with an anterior surgical approach. Each of the anterior and posterior components 712, 714 are configured to each be load bearing and may also be configured to mimic the anatomy of a disc. The posterior component 714 comprises an anterior side 751 and a posterior side 752 (see FIG. 11) and a body 711. Extending from the posterior side 751 of said body 711 is a first posterior extension body portion 754 and a second posterior extension body portion 755. Defined in said first and second extension body portions 754, 755 are receptacles 718. The body 711 also comprises a third posterior extension body portion 753 having a receptacle 719 defined therein. The first and second extension body portions 754, 755 extend from the anterior side 751 of the posterior body 711 at an angle generally orthogonal to the elongate axis X2 of the posterior component 714. While many configurations are contemplated, in most embodiments, the modular implant 700 is wider than it is high. First posterior extension member 722 and second extension member 724 extend from the posterior side 762 of the anterior component body 713 at an angle generally orthogonal to the elongate axis Xl of the anterior component 712. It should be noted that the elongate axis of a component typically relates to the largest dimension, length, width or height of the component body. If the component body is arcuate then the elongate axis is the vector which represents the largest diameter of the arcuate body. If the body comprises both arcuate and generally straight portions then the elongate axis represents the longest dimension that takes into account both straight and elongate body portions. The anterior component 712 also comprise a first channel 715, a second channel 716 and a third channel 717. The first channel 715 opens at the anterior side 709 of the anterior component 712 and communicates partially with the posterior side 762. It is contemplated that there may be variation in the vector of the channel. In the example of channel 717, the channel 717 opens on the anterior side 709 and is defined by a vector V1 that transverses a plane 783 of the top surface 781 of the component 712. (FIG. 9). Conversely, channel 716 opens on the anterior side 709 and is defined by a vector V2 that transverses a plane 784 of the bottom surface 782 (see FIG. 9). However, it will be appreciated that the holes 715, 717 may be angled such that vectors V1 and V2 pass into the cavities 720a, 720b by simply exiting the anterior portion via the posterior side 762 alone, thereby avoiding the upper or lower load bearing portions of the implant and increasing the load bearing capacity accordingly.

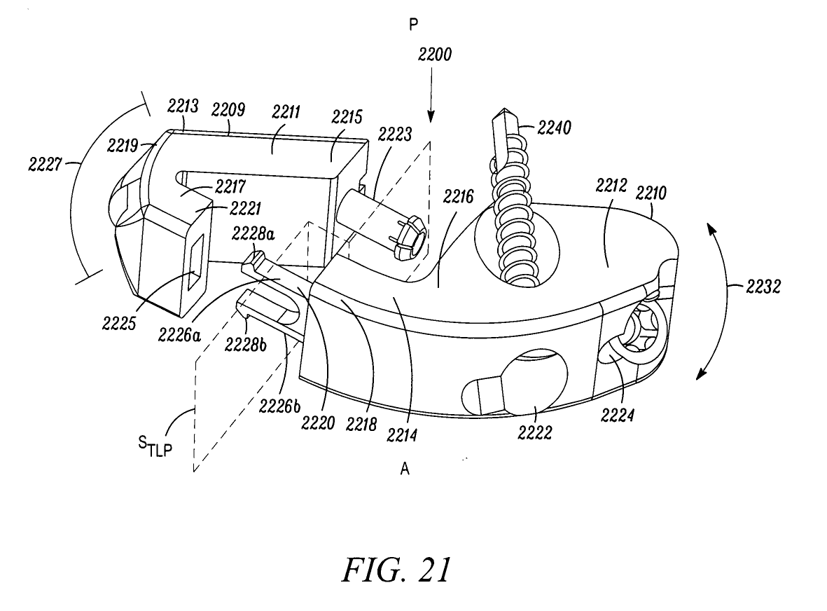

The anterior component 712 of the modular implant 700 comprises an anterior side 709 and a posterior side 762 (FIG. 10b) and a body 713. Extending from the posterior side 762 is a first extension member 722 and a second extension member 724 of FIG. 7. The first and second extension members are comprised of two arms 766, 767 that are compressible toward each other. The arms 766, 767 each comprises a lipped flange 768 defined at their posterior end. The anterior component also comprises an extension body portion 769 having a locking aperture 758 defined therein. An optional additional interlocking member 730, best seen in FIG. 8, passes through the interlocking aperture 758 and into receptacle 719. The interlocking member 730 may comprise a simple threaded screw engageable in a corresponding screw thread 721 on the interior of aperture 719. Alternatively, member 730 may comprise a sprung clip arrangement as shown at 722, 766, 724, 767 and 768 of FIG. 7. Still further, member 730 may comprise a bayonet fitting as shown generally at 2220 or 223 in FIG. 21.

FIG. 8 shows a perspective view of the modular implant 710 wherein the anterior component 712 and the posterior component 714 are adjoined. FIG. 8 also shows how cavities 720a, 720b are formed as the anterior component and posterior component are brought together. Also shown are projections 728 which are disposed on the top surface 781, 785 (see FIG. 11) and bottom surface 782, 786 (see FIG. 12) of the anterior component 712 and posterior component 714, respectively.

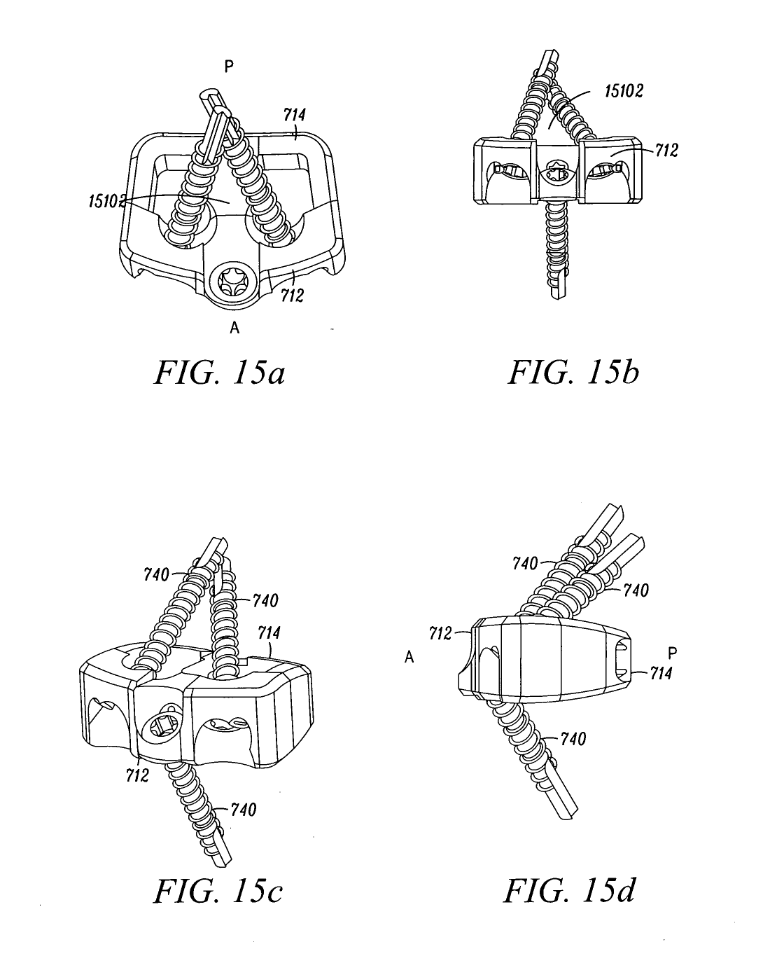

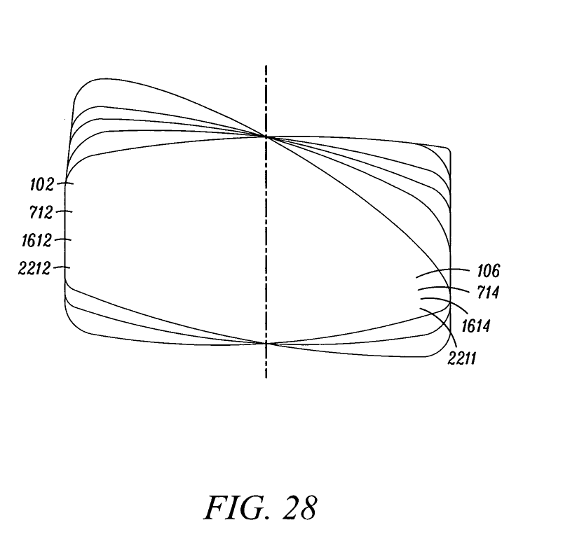

FIG. 9 shows an anterior to posterior side cross-section of the implant 10 shown in FIG. 8, taken along the A-A axis. The side cross-section shows a heightened portion B of the implant 710. The implant tapers C down to the posterior side and anterior side from the heightened portion, i.e., forming a tapered, convex shape. This and the shape of the implant in a sagittal plane (as shown in FIG. 15b) emulates the geometrically anatomy of a disc In preferred arrangements the implant is generally flat across the width thereof but it will be appreciated that in certain circumstances it can and may be convex or concave. Accordingly, another advantageous aspect of certain embodiments of the present invention is that the individual components are brought together and are designed such that regardless of the size of the individual components, they will be flush at there association line on the top and bottom surfaces. This design is achieved by making a coronal sectioning of the implant that occurs at the heightened portion and a variation thereof is explained with reference to FIG. 28 later herein. During surgery, typically trial spacers will be used to measure the disc space and lordosis. The anterior and posterior components are both configured to be load bearing and to restore disc anatomy, restore lordosis, and/or disc height. The arms 722, 724 are shown positioned into receptacle 718. The flanges 768 of arms 722,724 catch on ridge 736. This configuration snaps the anterior component 712 with the posterior component 714. As shown in FIG. 9, the posterior component 714 comprises an aperture 708 which exposes the ends of the arms 722,724 and against which open end said arms engage so as to lock the components together.

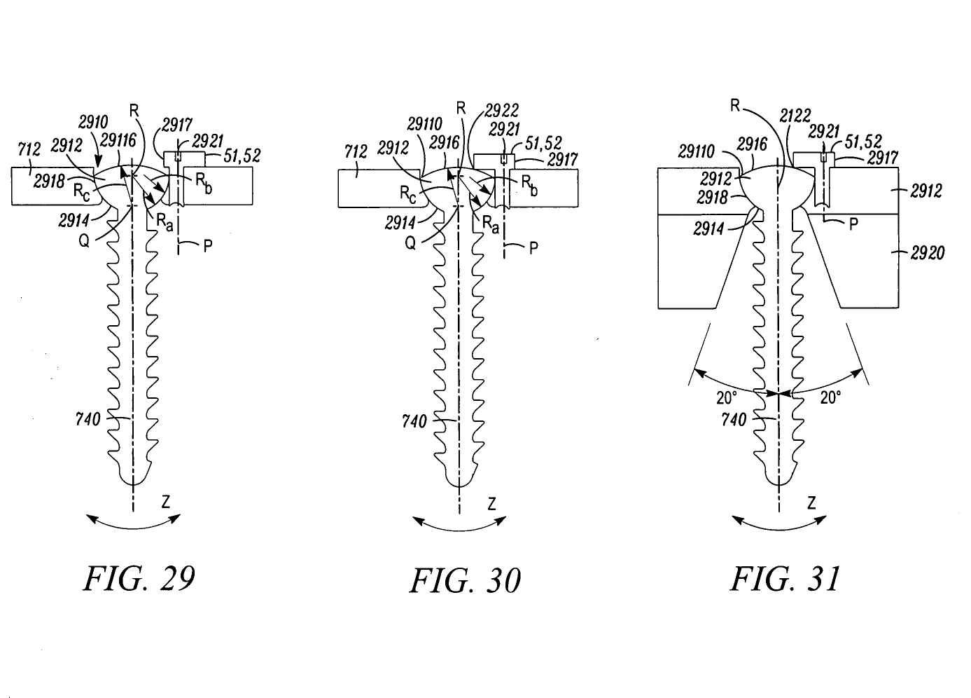

FIGS. 10 to 12 illustrate the implant in association with bone fixators shown generally at 740 and shown and discussed more particularly in FIGS. 29 to 31. These bone fixators may be employed with any one or more of the implants described herein and this description is, therefore, generic across all embodiments and for the reasons of brevity is not repeated in detail later herein. FIGS. 10 to 12 and 29 to 31 show a self-taping, self-drilling screw 740. The screw 740 comprises an enlongate body 741 comprising a proximal end 743 and distal end 745. The distal end 745 comprises a drill region 746 which is configured to initiate drilling a whole into bone. The elongate body comprises a taping region 747 which is configured to initiate taping into bone and a threaded region 744 which is configured to screw into bone.

The screw 740 and channels 715, 716, 717 are configured and sized such that the screw 740 passes through the channels 715, 716, 717 without engaging the channel wall. This allows for the screw, implant and vertebral body to be secured in an interfragmentary compression engagement to achieve a superior result. In a non-limiting preferred, the screw comprises a lag portion that rests against a portion of the channel wall. The channels 715, 716, 717 are configured such that the screw 740 comprises angular variability of 40 degrees or less, preferably 25 degrees or less, around a central axis of the respective channel. The central axis pertains to a vector V running through the center of the channel. As described above, the variability in the vector of the screw enables higher tolerances in screw placement and the avoidance of vital anatomical structures. FIG. 11 shows a posterior perspective view of implant 710 which shows the implementation of screws 740a, b, c. A driver 742 is defined in the proximal end of the screws 740. The driver may take many suitable forms such as a cross-head drive, flat head drive or it may be configured as a hex drive, as shown herein. The implant 710 comprises a perimeter side surface as designated by brackets and arrows.

FIG. 12 shows an anterior perspective view of implant 710 showing shiftable locking components 760a, b, c which serve to prevent backing out of screws 740a, b, c, respectively. The shiftable locking components serve to individually and separately lock the screws within the channels. FIG. 12 also shows the shiftable locking components 760 in a closed state. The shiftable locking components 760 are fixed to the anterior component 712 proximate to the channels 715, 717 such that they may be pivoted or otherwise shifted to cover the opening of the channels 715, 717, i.e., a closed state (see FIG. 12). The bone fixation device is described in detail with reference to FIGS. 29 to 31 later herein and, therefore, not repeated at this juncture.

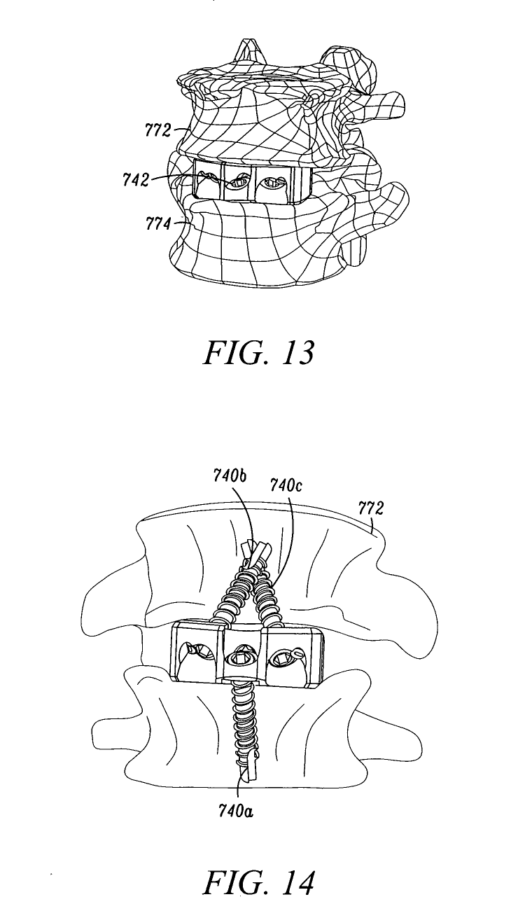

FIG. 13 shows the above implant secured to a superior 772 and inferior 774 vertebral body. The driver head of screw 740a is shown which has been turned to cause the screw 740 to penetrate the inferior verterbral body 774. FIG. 14 shows a see through perspective view of the implant 710. The implant 710 is secured to the superior vertebral body by screws 740 b and c and secured to the inferior vertebral body 774 by screw 740a. FIGS. 13 and 14 also illustrate the screw arrangements when filly engaged and from which it will be appreciated that when two or more screws are provided into the same vertebral body they may cross one an other, thereby to define therebetween a bolus or mass of vertebral body therebetween, as shown at 15102. Such an arrangement helps provide a more secure anchor and may prevent loosening of the screws or prevent them from simply pulling free when subjected to an otherwise excessive load. It will also be appreciated that inferior anchor 740a covers the head of locking bolt 730 and, therefore, prevents inadvertent backing out of said bolt.

FIG. 15 shows a top view 15a, a front view 15b, a side perspective view 15c and a side view 15 d of implant with screws positioned therethrough.

Examples 2-5 discussed below represent alternate embodiments of a modular implant useful in conjunction with an anterior surgical approach.

### EXAMPLE 2

### Anterior Approach

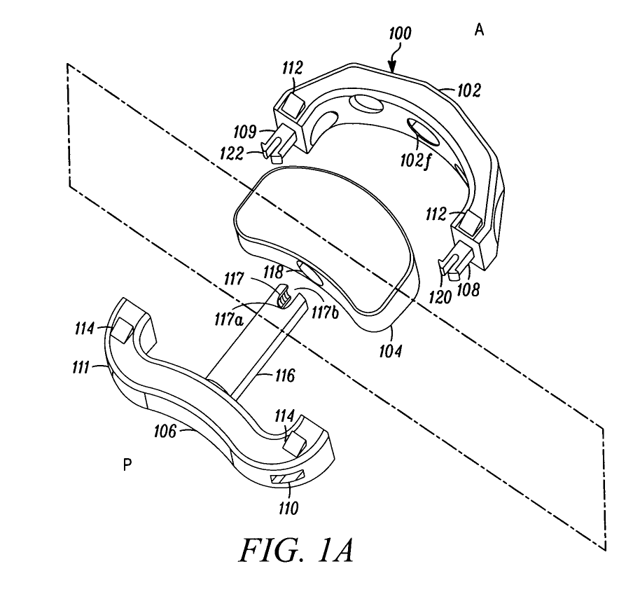

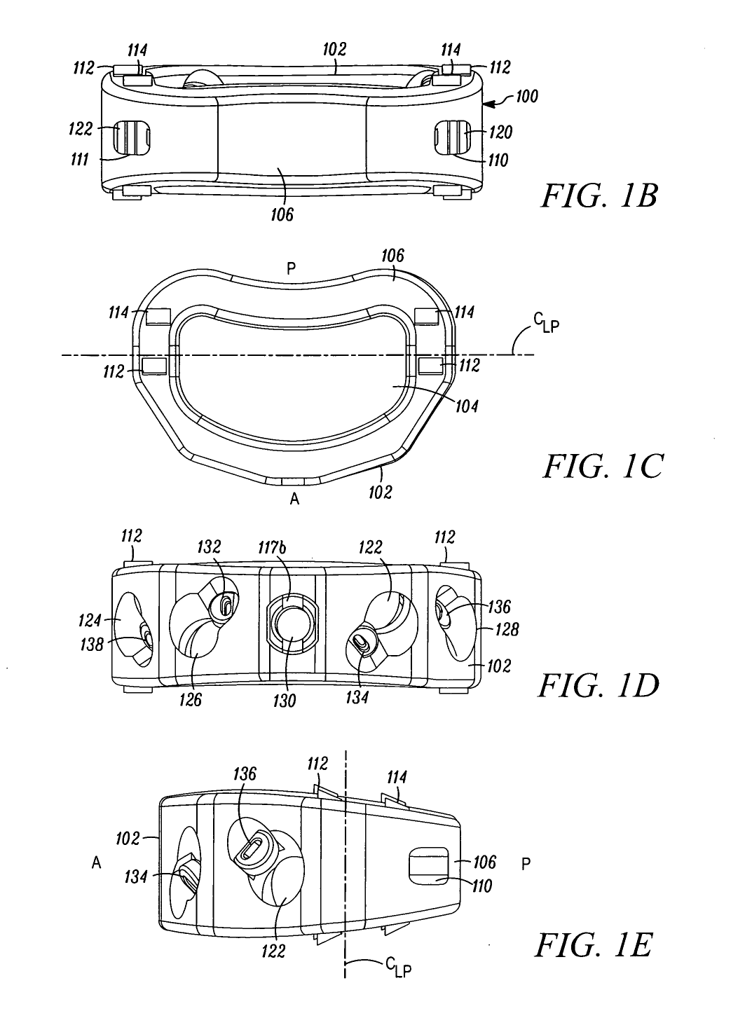

Turning now to FIG. 1, a disassembled, perspective view of a modular intrabody spinal implant embodiment is shown generally at 100. The implant 100 is sectioned along a coronal longitudinal plane C_{LP} and is particularly useful for implementation with an anterior surgical approach. The implant 100 comprises an anterior component 102, an optional core component 104, and a posterior component 106, which are brought together in an interlocking fashion either in Vivo or otherwise. Both the anterior component 102 and posterior component 106 are load bearing and will serve to restore anatomy, lordosis, and/or disc height when implanted, as will be discussed below. Male clasps 108, 109 are made from a resilient material and are associated with and extend from the anterior component 102 which are inserted into receptacles 110, 111 defined in the posterior component 106. Each clasp is provided with a lipped flange 120, 122 which engage with corresponding female lip portions provided in the corresponding receptacle 108, 109 such as to provide the arrangement with a “ckick-fit” as discussed below. Those skilled in the art will appreciate that the illustrated snap-fit design of the male clasps 108, 109 and receptacles 110,111 is only one type of clasping mechanism; several clasping mechanisms can be utilized to lock together the anterior and posterior components 102, 106, including, but not limited to, snap fit, friction fit, pin-in-screw, nut and bolt, etc.

The optional core component 104 may be secured into place via rod 116 extending from posterior component 106 which runs through channel 118 defined in core component 104 as the anterior component 102 and posterior component 106 are mated together. The anterior component 102 and posterior component 106 may comprise a gripping means 112, 114, respectively, which pushes into the superior and inferior vertebrae (not shown) to assist in keeping the implant 100 in place once properly placed in the spine. Rod 116 is provided with a location feature 117, the function of which will become apparent later herein and preferably includes a threaded section 117a.

FIG. 1B shows a posterior side view of the implant 100 as assembled whilst FIG. 1C shows a top view of the assembled implant 100. The core component 104 is shown secured in the space defined by the anterior and posterior components 102, 106. FIG. 1D shows an anterior view of the implant 100 which better displays the anti-backout mechanism. Channels 122, 124, 126, and 128 are defined in the anterior component 102 which allow the placement of fixators into the anterior component 102 through the core component 104 (when fitted) and through the superior and inferior vertebrae adjacent to the implant 100. As with embodiment 700, the channels are configured to allow for 40 degrees or less of angular variability. Proximate to at each channel is a lock component 132, 134, 136, and 138, as shown in FIGS. 1D and 1E and discussed in detail above with reference to FIG. 12 and to which the reader's attention is now drawn. Each of the lock components 132, 134, 136 and 138 are movably affixed to the anterior component 102 and proximate to at least one of said at least two channels such that said locking components 132, 134, 136, and 138 can be shifted to block at least a portion of a proximate channel. Each channel is individually and separately lockable. FIG. 1D also shows the edges of 117 and from which it will be appreciated that the slot 117b may be used for location purposes, as discussed later herein.

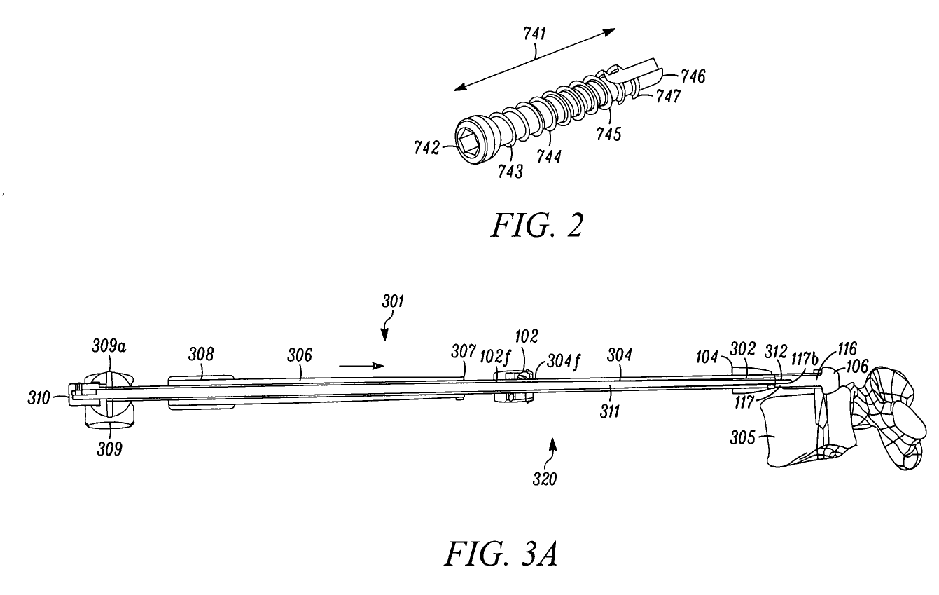

FIG. 2 shows a perspective view of one embodiment of a fixator in the form of a screw. The self drilling screw eliminates the new for use of an awl. Further, the universal head allows fixation of hex driver.

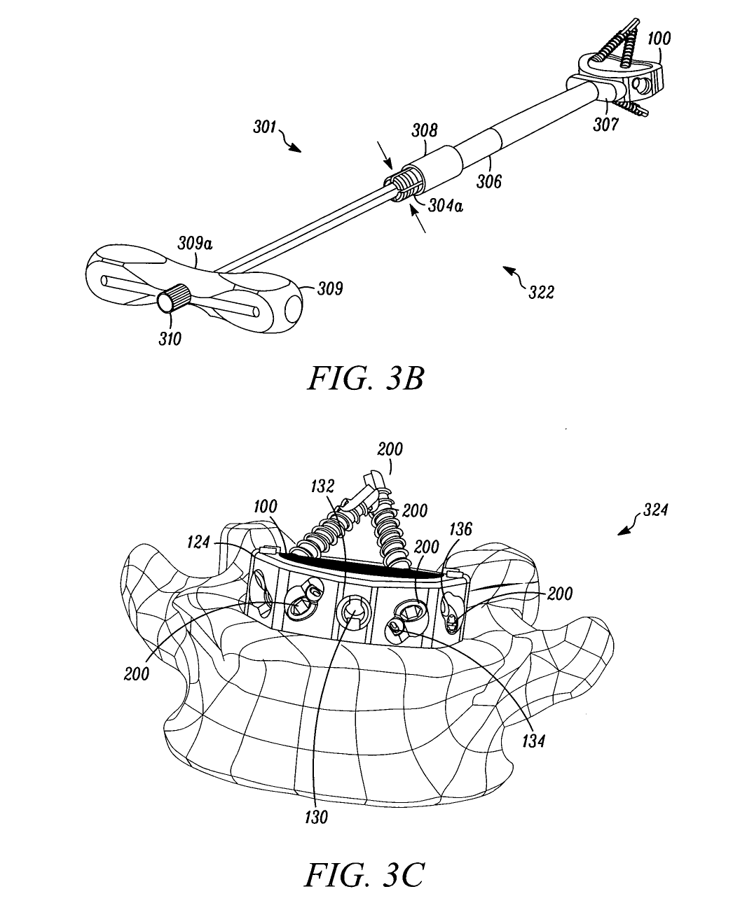

FIGS. 3A and 3B illustrate an implantation device suitable for implanting the implant 100 shown in FIG. 1 The device 301 comprises a first engagement member in the form of hollow shaft 304 having a location feature 302 for engagement with location feature 117 on the posterior portion 106 (best seen in FIG. 1a) and second engagement member in the form of an elongate anterior introducer sleeve 306 slidably engaged to the shaft 304 and being provided with location features 307 for engagement with the anterior portion 102. At the proximal end of the anterior introducer 306 is a locking collet 308, the function of which will be described shortly. A handle 309 is associated with shaft 304 by pins 309a at its distal end such that rotation or movement of the handle rotates or moves the shaft 304 and anything associated therewith. A posterior thread interface knob 310 is associated with an inner shaft 311 at its proximal end and includes a threaded portion 312 at a distal end thereof for locking engagement with the thread 117b on posterior portion 106 of the implant 100. The locking collet 308 is shown in more detail in FIG. 3B and from this drawing it will also be appreciated that the intermediate shaft 304 also includes a threaded end 304a split into segments 304b such as to create flexible fingers at the end of said portion. The inner surface of the collet is provided with corresponding tapers and threaded portion such that lateral displacement of the collar 308 towards handle 309 will tighten the segments 304b radially inwardly such as to engage with and lock against inner shaft 311. Preferably, the instrument further includes an alignment feature such as a flat 304f formed on said shaft 304 which matches a corresponding flat 102f or similar feature on a portion of an anterior portion and aligns said anterior portion with said posterior portion such as to allow an anterior portion to be slid along said shaft 304 whilst maintaining said alignment such that the interlocking features on the anterior and posterior portions are aligned before final securement of the two portions to each other. It will be appreciated that the threaded portion 312 may be replaced by a simple twist lock or the like and that the flat may be replaced by a keyway or the like.

In a first step of a method embodiment 320, the core component 104 with posterior component associated therewith 106 is placed onto implantation device 301 and inner shaft 311 is engaged with the posterior portion 106 by inserting the location feature 302 into the end of 116 such as to engage thread 117a and lock the components together. In a second step of the method 322 shown in FIG. 3B, the timed anterior introducer 306 is slid distally which pushes the anterior component 102 into the surgical site and mates together the posterior component 106, core component 104 and anterior component 102. The various “click-fit” components engage automatically during assembly and act to secure the components together. Once the assembled implant 100 is in place, 4 fixators 200 (superior fixators exposed), 2 superiorly and 2 inferiorly, are put through the channels 122, 124, 126 and 128 (124 obliquely shown) and secured in adjacent vertebra in a third step 324 shown in FIG. 3C. The inner shaft is then turned via the posterior threaded interface knob 310 such as to disengage the device from the implant and removed before inserting optional retaining screw 730, described above. Optionally, disengagement may be done before the fixators are inserted. Should it be necessary or desirable, the surgeon may lock the outer sleeve 306 to the intermediate shaft 304 such as to allow him to move all components as one. This can be particularly useful if it is difficult to place the implant as more pressure can be applied to the locked assembly than might be applied to the individual components. In essence, the implantation device also acts as an assembly device, assembling the components either within the vertebral space or outside thereof and may be used for either purpose. Additionally, by virtue of the fact that the device is secured to the implant, the surgeon may use the device itself as a tool for the accurate and forceful insertion of an implant into what could otherwise be difficult locations.

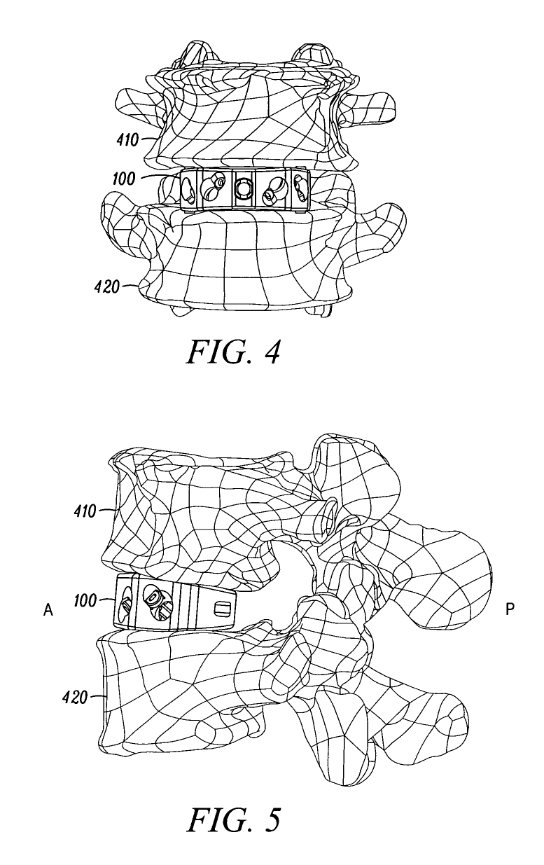

FIG. 4 shows the implant 100 from an anterior perspective fixated to a superior 410 and inferior 420 vertebral body. FIG. 5 shows a view of the implant 100 shown in FIG. 4 from a lateral perspective.

### EXAMPLE 3

### Anterior Approach

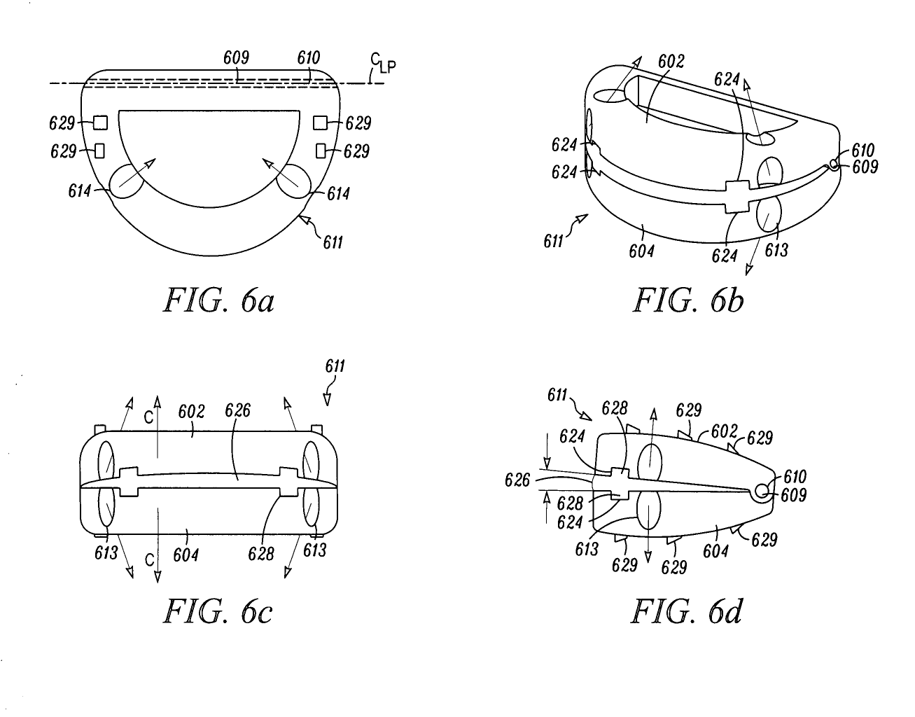

FIG. 6A shows a top view of an alternative angularly adjustable implant 611 and comprising inferior holes 613 and superior holes 614 depending on placement of implant 601. FIG. 6B shows a perspective view of said implant embodiment 661 having a superior component 602 and an inferior component 604 which are pivotally associated by a hinge 609 having a pin 610 extending in a coronal longitudinal plane C_{LP}. Embodiment 611 is particularly useful for an anterior surgical approach, but is unique to the other implants described herein as it is not sectioned along a longitudinal plane but rather it comprises upper and lower components adjoined at an edge by a hinge extending in the coronal plane. The pin may be made of PEEK, tantalum or other suitable material. FIG. 6C shows an anterior view of the implant embodiment 611 with superior and inferior components 602, 604 opened. Hinge 609 is configured so as to allow for height adjustment along the C-C vector such as to accommodate a wedge insert 626 within the gap formed therebetween. Such inserts may be of different sizes such as to allow the insert to be adjusted for height in the direction of arrows C. FIG. 6D shows a side view of the implant with the wedge inserted between components 602 and 604. The wedge insert 626 is angled 0 so as to correlate with the opening of components 602 and 604. The wedge insert 626 may also comprises pegs 628 which are inserted in peg holes 624 for stabilizing the wedge insert in the implant 661 and for ensuring load carrying capacity is provided between superior and inferior vertebra. Also shown are gripping means 629 to discourage slippage of the implant once placed in the surgical site. The implant 611 is secured to superior and inferior vertebra by means of fixation devices (not shown) positioned in the direction of arrows F throughout FIGS. 6a to 6d. Suitable fixation devices include those described with reference to fixation device 740 detailed in other portions of this document.

According to another embodiment, the subject invention pertains to a kit comprising the spinal implant 601 and a plurality of wedge inserts having different wedge angles. A wedge insert can be selected for assembly of an implant based on the anatomy and curvature of the patient's spine. In operation, the size of implant required is first determined by any suitable means before selecting the wedge size to suit. Once the appropriate wedge size is selected, the surgeon simply inserts the wedge within the implant such as to achieve the desired final height. This insertion may be done either in vivo or otherwise.

### EXAMPLE 4

### Lateral Approach

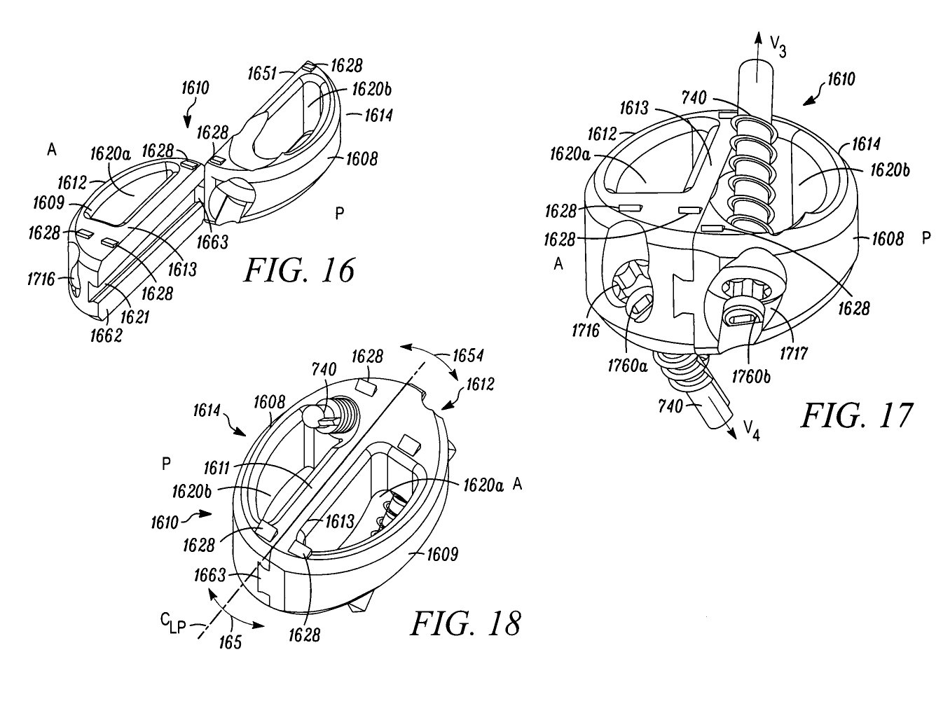

Another embodiment will now be described in reference to FIGS. 16-20. FIG. 16 shows a modular implant 1610 having an anterior component 1612 and a posterior component 1614. Implant 1610 is especially advantageous for use while implementing a lateral surgical approach. The anterior component 1612 and posterior component 1614 are sectioned along a coronal, longitudinal plane C_{LP}. The anterior component 1612 has a posterior side 1662 and posterior component 1614 has an anterior side 1651 which mate together. A groove 1621 is defined in the posterior side 1662 and a ridge 1663 projects out of the anterior side 1651 and is configured such that the ridge 1663 slides into groove 1621 during assembly. The ridge/groove configuration shown is a ‘dove-tail’ type but it is contemplated that other types could be implemented, such as, but not limited to, t-grove/ridge. Also, it is contemplated that more than one groove and ridge could be implemented on the respective anterior and posterior components 1612, 1614 and that the ridge/groove could be on either of the components. When provided, the dovetail or groove arrangement may comprise a tapered arrangement such that the gripping force between the components increases as they are pushed together, thereby to secure the components together once assembled. Anterior component 1612 has a body 1613 and posterior component also has a body 1611. An anterior side 1609 is integrated with body 1613 and a posterior side 1608 is integrated with body 1611. The sides 1608, 1609 and bodies 1611, 1613 define cavities 1620b and 1620a, respectively, into which bone ingrowth material can be disposed.

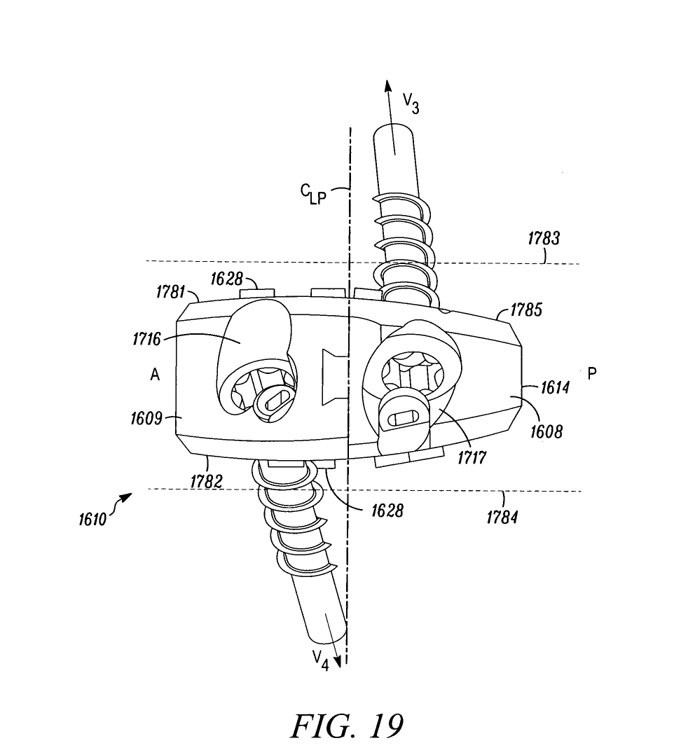

Referring now particularly to FIG. 19, anterior component 1612 has a channel 1716 which opens on the anterior side 1609. The channel 1716 is configured so as to be defined by a vector V4 in FIGS. 17 and 19 which begins at anterior side 1609 and transverse a plane 1784 of the bottom surface 1782 of the anterior component 1612. Posterior component 1614 has a channel 1717 which opens on the posterior side 1608. Channel 1717 is configured so as to be defined by a vector V3 which begins at posterior side 1608 and transverses a plane 1783 of the top surface 1785 of the posterior component 1614. The channels 1716, 1717 are configured to allow for a variability of 40 degrees or less, preferably 25 degrees or less around a central axis of the respective channels by screws 1640a and 1640b, respectively. The screws 1640a, b and channels 1716, 1717 are configured and sized such that the screws 1640a,b pass through the channels 1716, 1717 without engaging the channel wall. This allows for the screw, implant and vertebral body to be secured in an interfragmentary compression engagement to achieve a unified load path leading to a superior result. In a non-limiting preferred, the screw comprises a lag portion that rests against a portion of the channel wall. The anterior component 1612 and posterior component 1614 adjoin together to form a unitary implant having a first lateral end 1654 and a second lateral end 1655, a top surface formed by top surfaces 1781, 1785, a bottom surface formed by bottom surfaces 1782, 1786 and a side perimeter 1780 formed by sides 1609, 1608. Sides 1609, 1608 are shown as arcuate, but may be configured to have orthogonal regions. The implant top surface and implant bottom surface have projections 1628 which assist in gripping the implant 1610 to superior and inferior vertebral bodies. The reader's attention is drawn to the description of FIGS. 29 to 31 for a more detailed explanation of how the screw and channels are formed.

FIG. 17 also shows shiftable locking components 1760a, b, which serve to prevent backing out of screws 740a, b, respectively. Similar to that discussed above with reference to FIG. 12c, the shiftable locking components 1760a,b are fixed to the anterior component 1612 and posterior component 1614, respectively, proximate to the channels 1716, 1717 such that they may be individually and separately pivoted or otherwise shifted to cover the opening of the channels 1716, 1717. From FIG. 19 it will be appreciated that the two portions are adjoined along a Coronal longitudinal plane and at a point of common height approximately mid point between the anterior and posterior edges. This joining at a point of common height allows for the selection and adjoining of anterior and posterior portions of different overall heights and curvatures such as to more appropriately match or mimic the natural vertebreal disposition. Accordingly, another advantageous aspect of this and certain other embodiments of the present invention is that the individual components are brought together and are designed such that regardless of the size of the individual components, they will be flush at their association line on the top and bottom surfaces. This design is preferably achieved by making a coronal sectioning of the implant that occurs at the heightened portion. During surgery, typically trial spacers will be used to measure the disc space and lordosis. The anterior and posterior components are both configured to be load bearing and to restore disc anatomy, restore lordosis, and/or disc height. FIG. 29 later herein describes another variation on this approach.

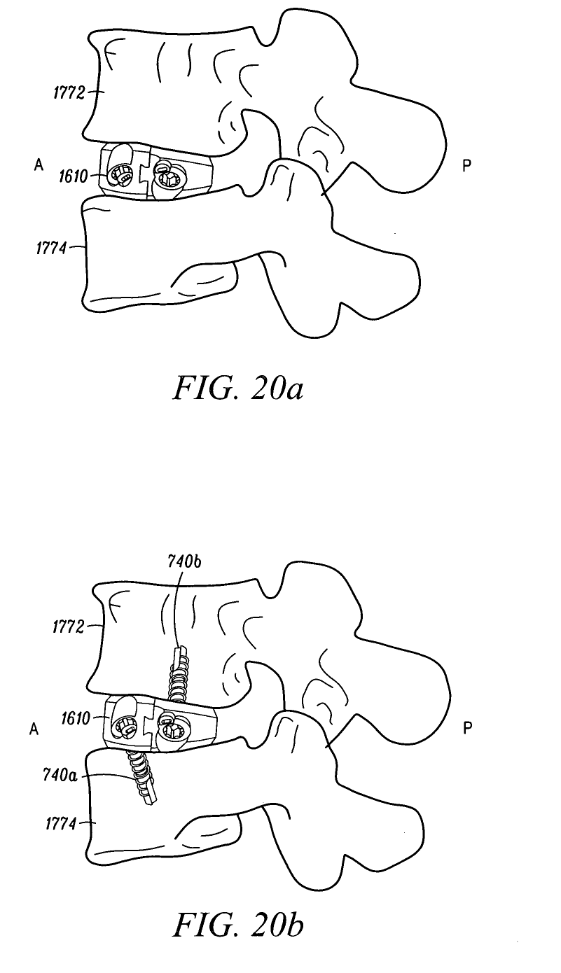

FIG. 20a shows the implant 1610 secured to a superior 1772 and inferior 1774 vertebral body. FIG. 2b0 shows a see through side view of the implant 1610. The implant 1610 is secured to the superior vertebral body 1772 by screw 740 b and secured to the inferior vertebral body 1774 by screw 740a.

### EXAMPLE 5

### Anterolateral Approach

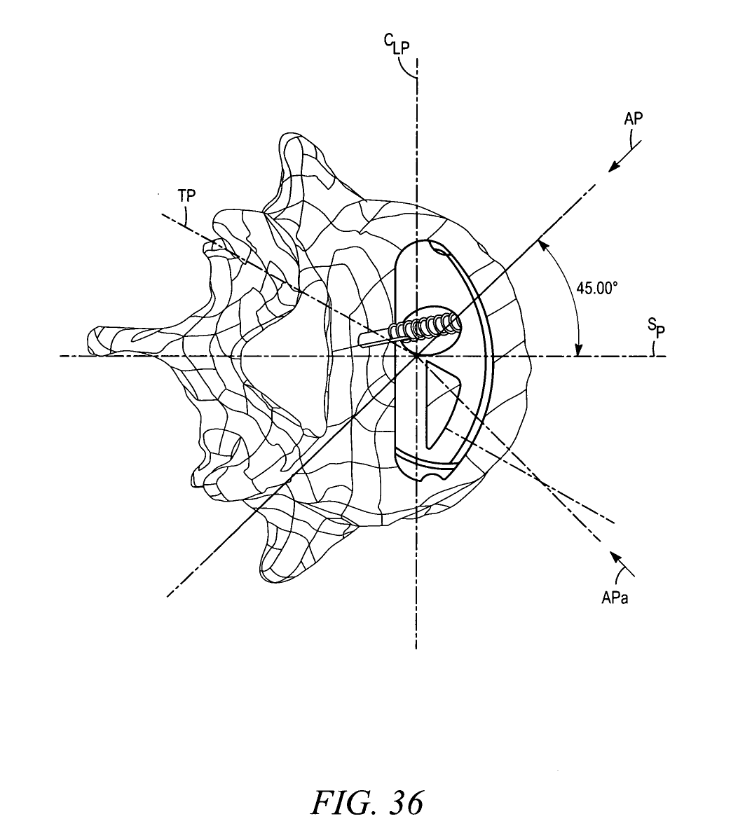

The following figures describe an implant comprising two portions assembled from an anterolateral surgical approach. The implant is split into two components, the first of which comprises a generally posterior component which extends in a lateral direction and the second component comprises a generally anterior component also extending in a lateral direction, as best illustrated in FIG. 36. It will be appreciated that the generally posterior component will have a small anterior portion and the generally anterior component will have a small posterior portion but for the purposes of brevity the components have been named to correspond with the adopted surgical approach.

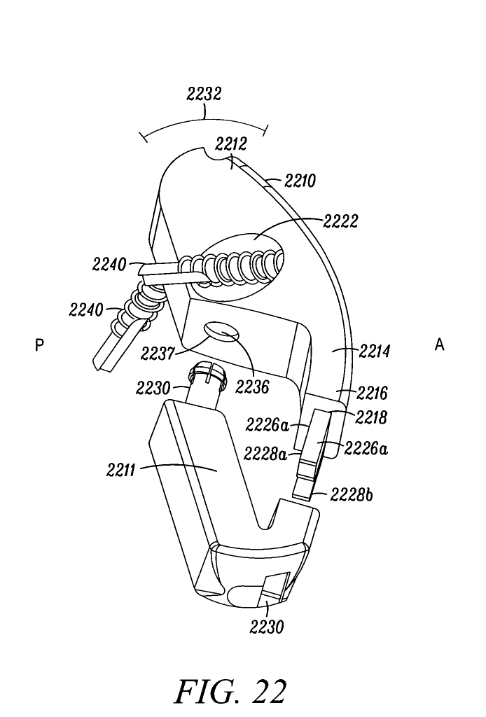

FIGS. 21 and 22 show, a modular interbody fusion implant 2200 in a disassembled state sectioned according to a stepped transverse, longitudinal plane, illustrated by dotted lines STLP and which is particularly useful in conjunction with an anterolateral surgical approach. The implant 2200 comprises a posterior lateral component 2209 and an anterior lateral component 2210. The posterior lateral component 2209 has a posterior body portion 2211 which has a lateral end 2213 and a medial end 2215. Extending from the medial end 2215 is an extension member 2223 having outwardly extending engagement members 2223a. The posterior lateral component 2209 also has a small anterior body portion 2217 having a lateral end 2219 and a medial end 2221. Defined through the medial end 2221 is receptacle 2225. The posterior body portion 2211 and the anterior body portion 2217 are joined at their lateral ends 2213 and 2219, respectively, to form a posterior component lateral end 2227.

The anterior lateral component 2210 has a generally lateral body portion 2212 and a medial body portion 2214 having a lateral end 2216 integrated (or otherwise associated with) body portion 2212. The lateral body portion 2212 also forms a lateral end 2232. Defined through the lateral body portion 2212 is a first channel 2222 (see dashed lines) and second channel 2224 (see dashed lines in FIG. 26). Positioned through channel 2222 is a screw 2240 similar to the screws shown in FIG. 11 discussed above. The screws 2240 and channels 2222, 2224 are preferably configured and sized such that the screws pass through the channels without engaging the channel wall. The screw may comprise a lag portion that rests against a portion of the channel wall. Furthermore, the channels 2222, 2224 are configured to allow for a variability of 40 degrees or less, preferably 25 degrees or less around a central axis of the respective channels by screws 2240

Extending from the medial end 2218 of the medial body portion 2214 is an extension member 2220.

The extension member 2220 has two arms 2226 a and 2226 b having locking flanges 2228 a and b, respectively. The two arms 2226 a and b are compressible toward each other. The arms are inserted into receptacle 2225 such that flanges 2228 appear from the other side thereof and spring outwardly to engage and lock the components together.

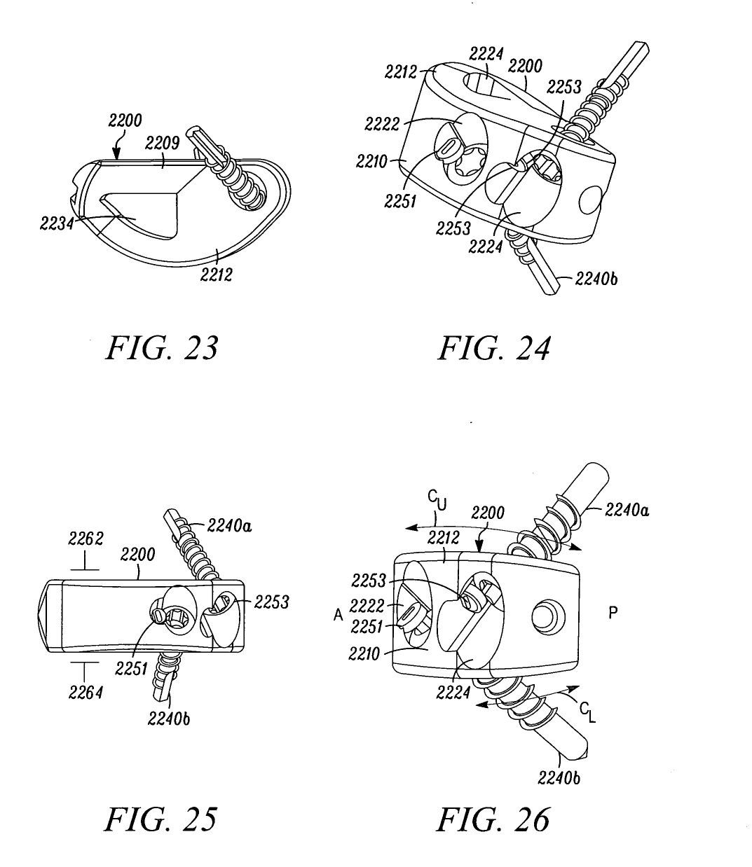

Defined on a medial side of the lateral body portion 2212 of FIG. 22 is a receptacle 2236 having a lip 2237 provided at an inlet thereto for receiving and engaging with engagement members 2223a of extension member 2223. The extension member 2223 is inserted into receptacle 2236 and extension member 2220 is inserted into receptacle 2225 to form a unitary implant as shown in FIG. 23. The posterior lateral component 2209 and the lateral body portion 2210 come together to form a cavity 2234 into which bone ingrowth material may be disposed. The assembled implant 2200 comprises a top surface (FIG. 25) 2262, a bottom surface 2264 and a side perimeter surface 2266.

FIG. 24 and FIG. 25 shows an anterior frontal view and front perspective view, respectively of the assembled implant. Shown also are shiftable locking components 2251, 2253 which serve to prevent backing out of screws 2240a and b, respectively. FIG. 24 shows the shiftable locking components 2251, 2253 in a closed state. The shiftable locking components 2251, 2253 are fixed to the anterior lateral component 2210 proximate to the channels 2222, 2224 respectively, such that they may be pivoted or otherwise shifted to cover the opening of the channels i.e., a closed state.

FIG. 26 shows the convex nature of the implant with arrows C_{U} C_{L} illustrating the extent of curvature across the top and bottom surfaces, 2262 2264 respectively. Similar to Examples 1 and 5, the implant tapers down from the anterior to the posterior side, thereby forming a tapered, convex shape. The general convex shape of the implant in a sagittal plane emulates the geometric anatomy of a disc. The anterior and posterior components are both configured to be load bearing and to restore disc anatomy, lordosis, and/or disc height.

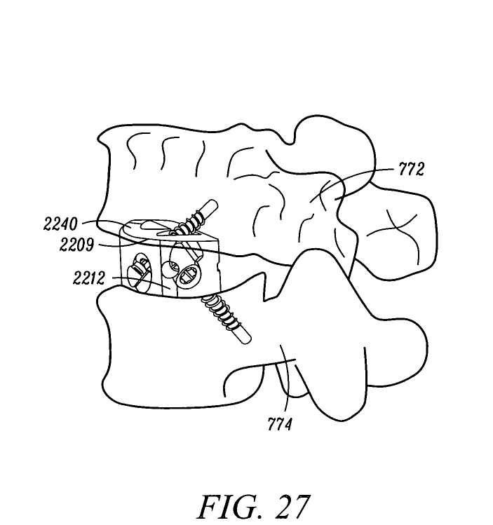

FIG. 27 shows a perspective view in a see-through fashion, the implant 2200 is secured to a superior 772 and inferior 774 vertebral body by screws 2240 which are turned to cause them to penetrate the vertebral bodies.

FIG. 28 is a diagrammatic representation of any of the implants described above split along a longitudinal plane LP and that the anterior portions 106, 714, 1614 or 2211 and the posterior portions 102, 712, 1612 and 2212 may be of various different ultimate heights and angles of taper and indeed surface shape and will mate together happily along the longitudinal plane without a step so long as they are at the same height at the join. This allows the surgeon to select anterior and posterior implant portions to suit a patients particular vertebreal support requirements in a manner that is not known in the art and which may well allow the surgeon to achieve better load carrying capacity than is presently known.

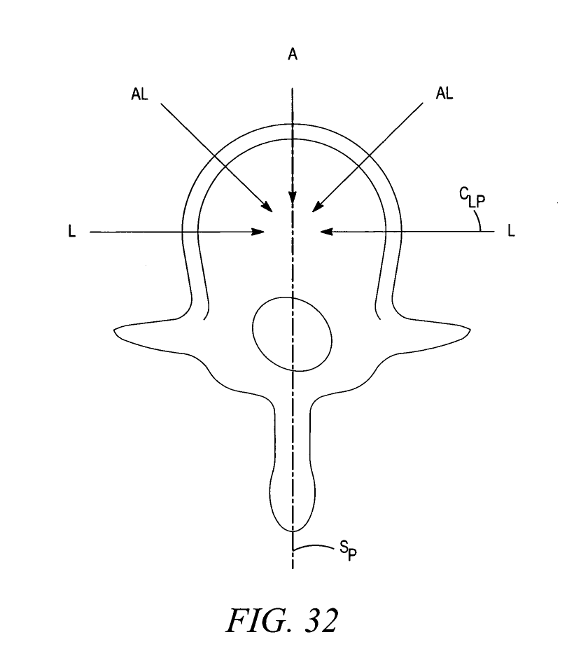

FIGS. 29 to 31 illustrates the bone fixation device 740 and locking components 51, 52 (also previously referred to as 132, 134, 136, 138, 760a, 760b, 1760a, 1760b, 2251 and 2253) in more detail and from which it will be appreciated that the locking component 51, 52 is rotatable about axis P between a first position shown in FIG. 29 where it acts to unobturate the channel 2910 and a second position shown in FIG. 30 where it acts to engage with the head 2912 and prevent the screw 740 from backing out of the channel 2910. The locking component shown comprises a generally circular component having a flattened side 2917 which acts to form an opening when rotated to a suitable position. A slot or other such feature 2921 may be provided for allowing a screwdriver or the like to engage with the lock and rotate it as and when desired. For further details please see the earlier figures. The screw head 2912 further includes a curved bottom surface 2914 having a radius Ra extending from point R and a curved top surface portion and having a radius Rc extending from point Q. The aperture itself is provided with an upper portion 2918 having a radius of curvature Rb matching or approximating that of Ra and an optional bottom portion 2920 (FIG. 31) which diverges, thereby to ensure adequate clearance for any angular movement of the screw 740. Radius Ra is selected such as to allow the screw 740 to pivot in the aperture whilst maintaining contact with the upper curved surface 2918. The upper curved surface 2912 is provided with a radius of curvature which may match that of the lower surface such that whenever the screw is pivoted the locking component 51, 52 will always be able to rotate into contact with the surface 2912 such as to cause said component to initiate a point contact at point 2922 and lock said locking component thereto such as to prevent movement of said screw out of said aperture. This is in contrast with the known art which merely acts to obdurate the aperture without actually engaging with the screw itself. It will be appreciated that radius Rc may be selected to be the same as radius Ra and that both may share a common origin such as to ensure a consistent and even clamping effect when the locking component 51, 52 is engaged with the head portion 2916. It will also be appreciated that the edge of the locking components 51, 52 may be modified to include a chamfered or curved edge at the point of contact with the screw head, thereby to increase the area of contact by making the contact a line contact rather than a point contact as commented upon above.

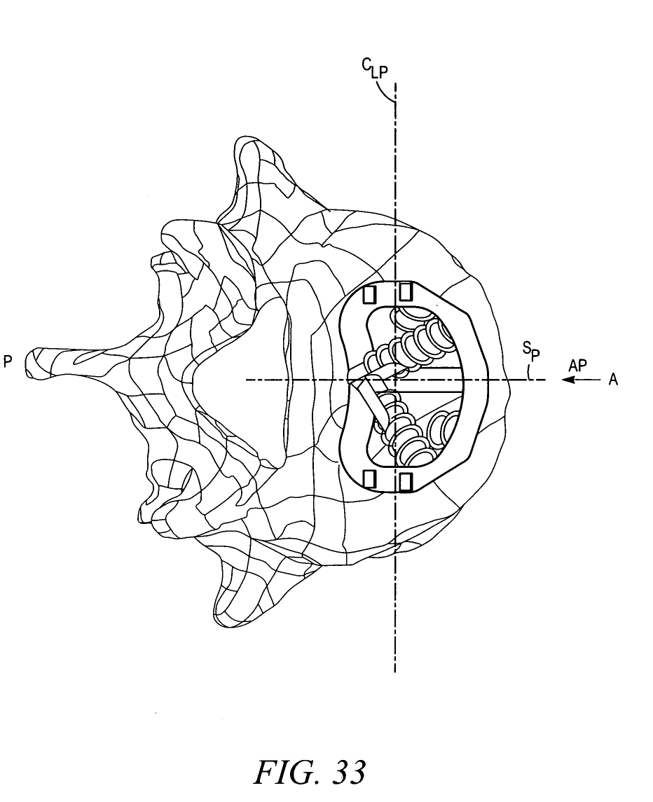

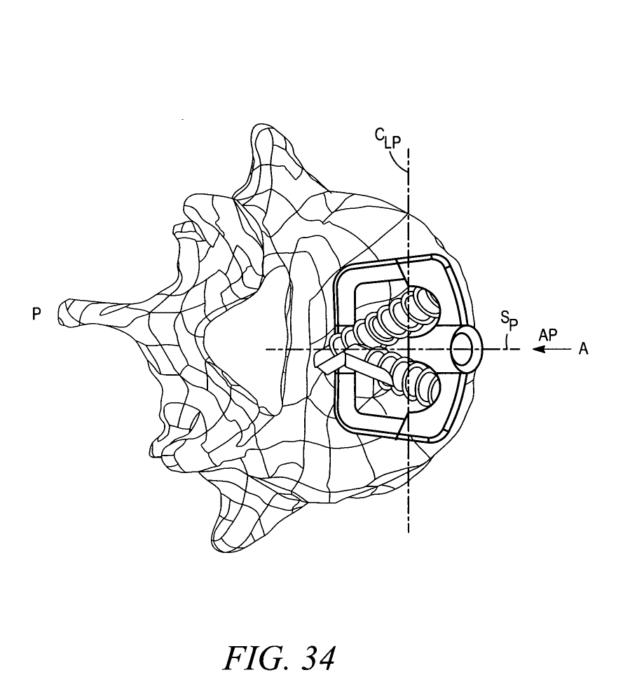

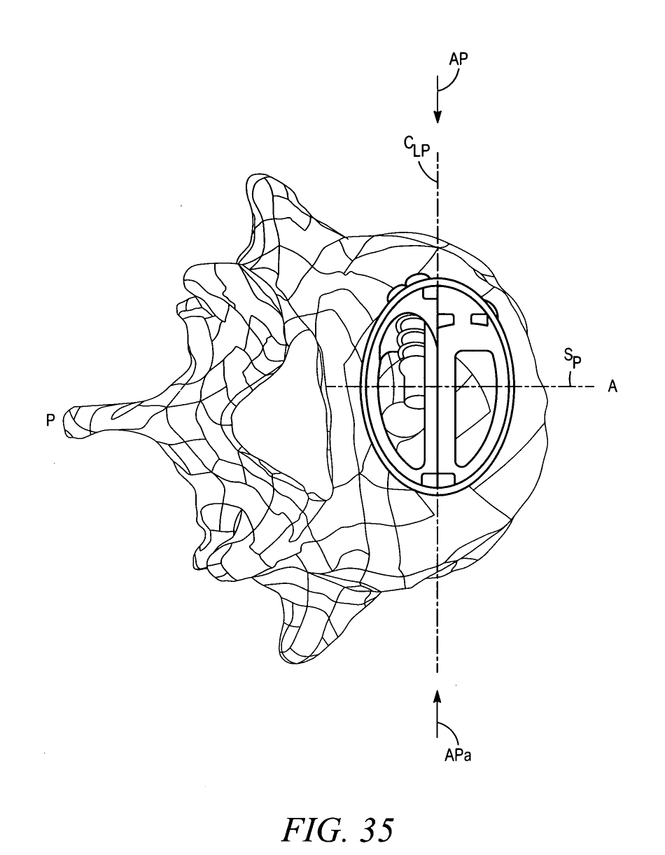

FIG. 32 is a plan view of a vertebra illustrating the different surgical approaches where A is anterior, AL is anteriolateral and L is lateral. FIGS. 33 to 36 illustrate each of the above-described embodiments of the present invention when positioned in the inter-vertebral cavity and how they relate to the Coronal Longitudinal Plane CLP and the Sagital Plane SP and wherein arrow AP indicates the approach angle and APa indicates an alternative approach angle (where applicable).

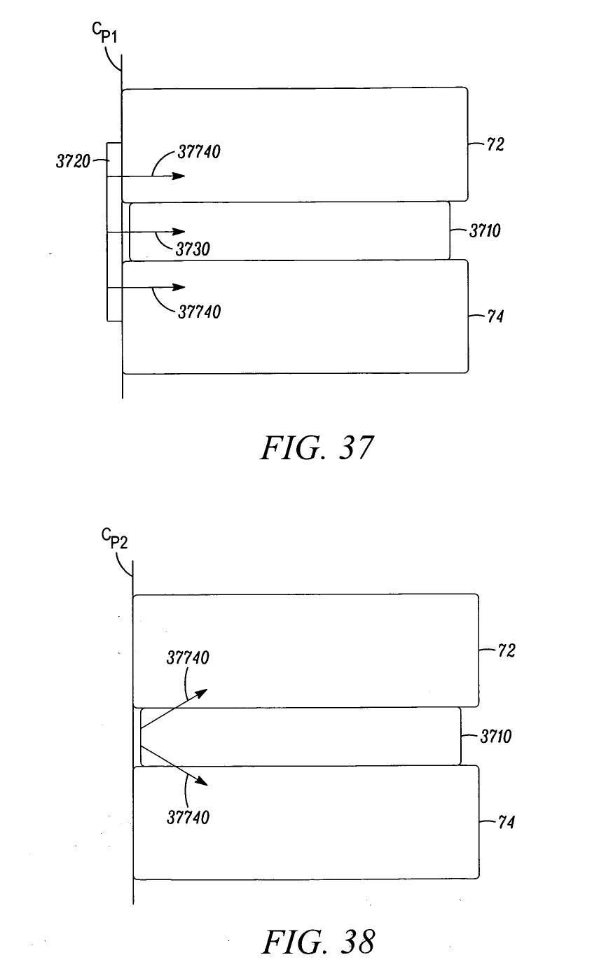

FIGS. 37 and 38 illustrate a cage arrangement well known in the prior art in which an implant shown generally at 3710 is secured in position by a relatively low profile plate 3720 provided on the outside of the vertebra and bridging two adjacent vertebra 3772 and 3774 such as to prevent the implant from migrating out of the inter-vertebral gap. The plate may be secured by screws shown at 37740 and may also be secured to the implant by means of a screw or other such device shown schematically at 3730. Whilst such an arrangement does not provide a “no-Profile” method of securing an implant it can be adequate in some circumstances and may lend itself to use with the present arrangements where the screw 2730 is secured to the implants of the present invention, thereby avoiding or supplementing the use of screws 740 of the above arrangements. It will, therefore, be appreciated that screws 740 may be eliminated in some circumstances and are important but not absolutely essential to the presently described inventive concept. FIG. 38 by contrast illustrates the arrangement of the present invention when secured to the vertebral bodies and from which it will be appreciated that it can provide a truly “no profile” method of securing an implant which reduces and possibly eliminates the problems of the prior art arrangements.

### Implant Materials

Embodiments of the present invention may implement various bioactive and biocompatible implant materials for making the implant components. In exemplary embodiments, the materials used are capable of withstanding large dynamic, compressive loads, encountered in the spine. Moreover, the implant materials used with embodiments of the present invention may implement radiopacity materials known in the art.

In some embodiments, the materials for making components of a implant are comprised of a biocompatible, hardenable polymeric matrix reinforced with bioactive and non-bioactive fillers. The materials can be comprised of about 10% to about 90% by weight of the polymeric matrix and about 10% to about 90% by weight of one or more fillers. The materials can also be comprised of about 20% to about 50% by weight of the polymeric matrix and about 50% to about 80% by weight of one or more fillers. In order to promote bone bonding to the implants, the implants of the present invention can be comprised of a bioactive material that can comprise a polymeric blended resin reinforced with bioactive ceramic fillers. Examples of such bioactive materials can be found, for example, in U.S. Pat. Nos. 5,681,872 and 5,914,356 and pending U.S. application Ser. No. 10/127,947, which is assigned to the assignee of the present invention and incorporated herein by reference in its entirety.

Also discussed herein is the use of bone ingrowth materials which are disposed within the various cavities of the embodiments, and/or used as coating the components. Further, in alternate embodiments, bone ingrowth materials are used for making the actual structural components. Bone ingrowth materials may comprise known bioactive materials including but not limited to BMP or other suitable growth factors, allograft bone with/without stem cell enrichment, calcium phosphate, and/or autograft bone. See U.S. Pat. Nos. 6,899,107 and 6,758,849 for general information on osteoinductive, osteoconductive and/or osteogenic materials and implants.

The disclosures of the cited patent documents, publications and references are incorporated herein in their entirety to the extent not inconsistent with the teachings herein. It should be understood that the examples and embodiments described herein are for illustrative purposes only and that various modifications or changes in light thereof will be suggested to persons skilled in the art and are to be included within the spirit and purview of this application.

It will be appreciated that the above described implants are easily assembled in vivo or otherwise and that the “click-fit” approach ensures security of assembly once assembly is completed and eliminates the requirement for separate screw type securing devices. Additionally, the fact that the implants are split/adjoined along a plane allows for easy and rapid assembly and allows for the creation of a kit-of-parts which can accommodate different sized anterior and posterior portions. Still further, the fixation devices 740 may be secured with a freedom of positioning not hithertobefore known whilst the locking mechanism ensures that they stay in place once secured.

1-80. (canceled)

81. A modular interbody implant comprising
a first component comprising a perimeter side surface, a top surface, a longitudinal plane and a bottom surface; and
a second component comprising a perimeter side surface, a top surface, a longitudinal plane and a bottom surface, said first and second components being adjoined together along said longitudinal plane to comprise a unitary implant comprising an implant top surface, an implant bottom surface and an implant perimeter side surface and wherein said first and second components form a tapered dimension in a sagital plane of said unitary implant thereby emulating a disc anatomy.

82. A modular interbody implant as claimed in claim 81, wherein said unitary implant comprises a first channel being defined according to a vector that begins at said implant perimeter side surface and traverses a plane of said implant top surface and a second channel being defined according to a vector that begins at said implant perimeter side surface and traverses a plane of said implant bottom surface.

83. A modular interbody implant as claimed in claim 81 wherein said first and second components form a tapered convex form in a sagital plane and an elliptical dimension in a coronal plane.

84. The implant of claim 81, wherein:
(i) said second component comprises at least two channels defined therethrough, said at least two channels comprising at least one channel being defined according to a vector that begins at said implant perimeter side surface and traverses a plane of said implant top surface and at least one channel defined according to a vector that begins at said implant perimeter side surface and traverses a plane of said implant bottom surface; or
(ii) said first component comprises at least one channel defined therethrough defined according to a vector that begins at said implant perimeter side surface and traverses a plane of said implant top surface or implant bottom and said second component comprises at least one channel defined therethrough defined according to a vector that begins at said implant perimeter side surface and traverse a plane of said implant top surface or implant bottom surface.

85. The implant of claim 81, wherein said first and second channels are sized and configured such that an elongate bone fixator may separately pass through each of said at least two channels so as to allow for up to 20 degrees either side of centre angular variability of said elongate bone fixator about a central axis of each of said first and second channels.

86. The implant as claimed in claim 85 wherein one or more of said first and second channels comprise tapered channels.

87. The implant of claim 86, wherein said first and second channels are configured so as to allow an interfragmentary association with said elongate bone fixator.

88. The implant of claim 81, wherein said implant comprises an instrument interface associated therewith.

89. The implant of claim 88, wherein said instrument interface is an interface receptacle defined in said unitary implant or an interface extension extending from said implant.

90. The implant of claim 82 further comprising at least one locking component movably affixed thereto and proximate to at least one of said at least two channels such that said locking component can be shifted to block at least a portion of said proximate channel.

91. The implant of claim 81 useful for an anterior surgical approach, wherein:
the first component is a posterior component (PC) comprising a PC perimeter side surface, a PC top surface and a PC bottom surface; and
the second component is an anterior component (AC) comprising an AC perimeter side surface, an AC top surface and an AC bottom surface, wherein:
(i) said anterior and posterior components are adjoined together along a coronal, longitudinal plane to form a unitary implant comprising an implant perimeter side surface having an implant anterior side and an implant posterior side, a first implant lateral side extending in a direction between said implant anterior and posterior sides and a second implant lateral side extending in a direction between said implant anterior and posterior sides; and an implant top surface and an implant bottom surface;
(ii) said implant anterior side is comprised of said anterior component and said posterior side is comprised of said posterior component.

92. The implant of claim 91 wherein said anterior component comprises at least one receptacle defined therein or at least one extension member, or both and said posterior component comprises at least one receptacle defined therein or at least one extension member; wherein said anterior component and posterior component are adjoined by mating of an extension member of one component with a receptacle in another component.

93. The implant of claim 91, wherein said anterior component comprises at least two channels defined therethrough, said at least two channels comprising at least one vector that beguins at said AC perimeter side surface and transverses a plane of said AC top surface and at least one channel defined by a vector that beguins at said AC perimeter side surface and traverses a plane of said AC bottom surface.

94. The modular interbody implant of claim 81 useful for an antero-lateral surgical approach, wherein:
said first and second components are adjoined together along a transverse longitudinal plane to form a unitary implant comprising an implant perimeter side surface an implant top surface and an implant bottom surface; and, optionally,
said second component comprises said first and second channels or said second component comprises at least one channel and said first component comprises at least one channel.

95. The modular interbody implant of claim 94, wherein said unitary implant defines a cavity contained within at least a majority of said implant perimeter side surface, said cavity communicating with said implant top surface or said implant bottom surface, or both.

96. The implant of claim 94, wherein said implant comprises at least one locking component movably affixed thereto and proximate to each of said first and second channels such that said locking component can be shifted to block at least a portion of its proximate channel.

97. The implant of claim 94, wherein said first and second channels are sized and configured such that an elongate bone fixator may separately pass through each of said at least two channels so as to allow for up to 20 degrees either side of centreangular variability of said elongate bone fixator about a central axis of each of said first and second channels.

98. The implant of claim 97, wherein said first and second channels are configured so as to allow an interfragmentary association with said elongate bone fixator.

99. The modular interbody implant of claim 81 for use with a lateral surgical approach, wherein:
the first component is a posterior component (PC) comprising a PC body having a PC anterior side
the second component is an anterior component (AC) comprising an AC body having an AC posterior side; wherein:
said anterior component and said posterior component are adjoinable together along a coronal, longitudinal plane such that said AC posterior side and said PC anterior side face each other to form a unitary implant having an implant perimeter side surface having first lateral end, an implant second lateral end, an implant anterior side, and an implant posterior side, and an implant top surface and an implant bottom surface.

100. The modular interbody implant of claim 99 wherein said AC posterior and said PC anterior side are slidably or otherwise engaged.

101. The modular interbody implant of claim 99, wherein said AC posterior side comprises a groove and PC anterior side comprises a ridge member, or vice versa, wherein said ridge member is configured to slide and lock into said groove.

102. The implant of claim 99 wherein said first and second channels are sized and configured such that an elongate bone fixator may separately pass through each of said at least two channels so as to allow for up to 20 degrees either side of centre angular variability of said elongate bone fixator about a central axis of each of said first and second channels.

103. The implant of claim 102, wherein said first and second channels are configured so as to allow an interfragmentary association with said elongate bone fixator.

104. The implant of claim 99 wherein said anterior component comprises at least one of said first and second channels and said posterior component comprises at least one of said first and second channels.
